(19) United States
(12) Patent Application Publication
(10) Pub. No.: US 2026/0250730 A1
(43) Pub. Date: Aug. 27, 2026

(54) GENETICALLY MODIFIED MICROBIAL STRAIN WITH INCREASED TOLERANCE TO ISOPRENOL

(71) Applicant: THE REGENTS OF THE UNIVERSITY OF CALIFORNIA, Oakland, CA (US)

(72) Inventors: Hyungyu LIM, San Diego, CA (US); Adam FEIST, San Diego, CA (US); Aparajitha SRINIVASAN, San Francisco, CA (US); Aindrila MUKHOPADHYAY, San Francisco, CA (US)

(21) Appl. No.: 19/441,666

(22) Filed: Jan. 6, 2026

Related U.S. Application Data
(60) Provisional application No. 63/763,884, filed on Feb. 26, 2025.

(51) Int. Cl. C12P 17/12; C12N 15/78; C12P 7/04

(52) U.S. Cl. CPC C12P 17/12; C12N 15/78; C12P 7/04; C12N 2800/101

(57) ABSTRACT

The present invention provides for a genetically modified microbial strain capable of growing or dividing in a medium comprising equal to or more than about 4.5 g/L isoprenol, or having an increased tolerance to isoprenol compared to a unmodified microbial strain.

### CROSS REFERENCE TO RELATED APPLICATIONS

This application claims priority to U.S. Provisional Patent Application Ser. No. 63/763,884, filed Feb. 25, 2025, which are hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of strain tolerance to isoprenol.

### REFERENCE TO A “SEQUENCE LISTING” SUBMITTED AS XML FILE VIA EFS-WEB

The instant application contains a Sequence Listing which has been submitted electronically in XML format and is hereby incorporated by reference in its entirety. Said XML copy, created on Jan. 6, 2026, is named “2024-092-02 Sequence Listing.xml” and is 19,581 bytes in size.

### BACKGROUND OF THE INVENTION

Isoprenol (3-methyl-3-buten-1-ol) is an established advanced biofuel and a precursor for diverse commodity chemicals (Zheng et al., 2013), and can be produced from biomass using heterologous pathway expression in a microbial host. Chemical catalysis can efficiently convert isoprenol to isoprene, a key component for synthetic rubber synthesis and more broadly, a drop-in platform chemical compatible with existing chemical manufacturing workflows (Emelianov et al., 2024; Kant et al., 2023). As a fuel molecule, it has a higher energy density compared to ethanol (36.5 MJ/kg vs 36.8 MJ/kg) (Pang et al., 2021). Moreover, it can be converted into 1,4 -dimethylcyclooctane (DMCO), an emerging sustainable aviation fuel (Baral et al., 2021; Benavides et al., 2022; Rosenkoetter et al., 2019). DMCO has a 9.2% higher volumetric net heat of combustion compared to that of the currently used JET-A (Baral et al., 2021; Rosenkoetter et al., 2019). Currently, the wide adoption of isoprenol is limited as existing bioconversion processes lack the high titers, rates, and yields to derisk its production at scale using renewable carbon streams.

Pseudomonas putida is a versatile microbial chassis with rapid growth, broad substrate utilization (e.g., coumarate, ferulate, benzoate), and high-stress tolerance, making it ideal for bioprocesses using biomass-derived carbon streams (Belda et al., 2016; Nikel and de Lorenzo, 2018; Wada et al., 2021). Targeted host engineering of P. putida has allowed efficient utilization of both five and six carbon sugars including xylose, arabinose, and galactose (Elmore et al., 2020; Lim et al., 2021; Mohamed et al., 2020). Furthermore, a range of systems biology approaches have enabled a better understanding of its transcriptional regulatory network (Borchert et al., 2024; Lim et al., 2022) informing new strain designs with sophisticated tools for genetic engineering (Martin-Pascual et al., 2021; Nikel et al., 2014). Currently, P. putida has been demonstrated as a viable host for a range of biochemicals including PHA (Cha et al., 2020), lycopene (Hernandez-Arranz et al., 2019), indigoidine (Banerjee et al., 2020; Eng et al., 2023; Gauttam et al., 2023; Lim et al., 2021), muconic acid (Almqvist et al., 2021; Bentley et al., 2020; Ling et al., 2022; van Duuren et al., 2011), lactic acid (Zou et al., 2021), adipic acid (Ackermann et al., 2021; Niu et al., 2020), medium-chain fatty acid methyl esters (Valencia et al., 2022) and more recently, isoprenol (Banerjee et al., 2024; Wang et al., 2022).

One critical challenge in using P. putida as an isoprenol production platform is product toxicity, despite its general tolerance to other commodity chemicals. Although it is known to exhibit relatively higher tolerance to hydrophobic compounds than model bacteria including Escherichia coli, cell growth is still inhibited since alcohols including isoprenol denature proteins and increase membrane instability (Huffer et al., 2011). Tolerizing microbes for high concentrations of a shorter alcohol, ethanol, has resulted in the identification of both degradative routes (i.e. increased or new enzymes capable of catabolism) and non-degradative routes (i.e., membrane fluidity, cell wall modifications, induction of stress response systems, chaperones, efflux pumps and production of compatible solutes such as glycine-betaine) (Mukhopadhyay, 2015). While previous reports for improved heterologous isoprenol production in P. putida exceed 1 g/L (Banerjee et al., 2024), product toxicity may limit further increases as intracellular isoprenol concentrations may approach growth inhibitory levels.

To improve tolerance phenotypes of microbial hosts, Adaptive Laboratory Evolution (ALE) has been widely applied (Sandberg et al., 2017). After continuous exposure to an increasing target inhibitory compound (a specific methodology referred to as Tolerization ALE or TALE) isolates with improved tolerance can be obtained. In addition, subsequent-omics investigations (e.g., genome, transcriptome, or proteome analysis) can suggest tolerization mechanisms, expanding our understanding about microbial physiology. Accordingly, there are many studies which have tolerized microorganisms against diverse growth inhibitors including alcohols (Halle et al., 2023), ionic liquids (Lim et al., 2020; Mohamed et al., 2017), and organic acids (Lennen et al., 2023; Nguyen-Vo et al., 2019). A recent evolution study with E. coli K-12 MG1655 has successfully increased the half-maximal inhibitory level against isoprenol by 47% (Babel and Krömer, 2020) and identified tolerance mechanisms with a subsequent mutation analysis. Further, P. putida can inherently catabolize isoprenol (Thompson et al., 2020), which could be one of the major tolerance mechanisms. To develop P. putida strains suitable for high isoprenol production, it is therefore likely important to explore tolerance mechanisms other than catabolism.

### SUMMARY OF THE INVENTION

The present invention provides for a genetically modified microbial strain capable of growing or dividing in a medium comprising equal to or more than about 4.5 g/L isoprenol, or having an increased tolerance to isoprenol, isopentanol, and/or prenol compared to a unmodified microbial strain, wherein the microbial strain comprises one or more mutations from the following gene/genetic region: gnuR (PP_3415), pcaK (PP_1376)-opdH, PP_3024-PP_5558, erdR (PP_1635) or mxtR (PP_1695), oprD (PP_1206)/PP_1207, infC (PP_2466), astA-II (PP_4480), topA (PP_2139), PP_2402-calA (PP_2426), PP_3595-PP_3610, rpoZ (PP_5301), and/or fleQ, and any gene/genetic region described herein, such as in Table 1.

TABLE 1
Commonly Mutated Isoprenol Tolerance Specific Regions.

| | | | Mutation | # of | ALE |
|---|---|---|---|---|---|
| # | Genetic region^{a} | Product | type | ALE | condition |
| 1 | gnuR (PP_3415)^{a} | Transcriptional regulator, LacI family | SNP, in-frame | 12 | A1-4, A5-8, |
| | | | deletion, frame- | | A9-12 |
| | | | shift deletion, | | |
| | | | early | | |
| | | | termination | | |
| 2 | pcaK (PP_1376)- | 22 Genes | Deletion | 11 | A1-4, A5-8, |
| | opdH^{a} | | | | A10-12 |
| 3 | PP_3024-PP_5558 | 53 Genes | Deletion | 6 | A1, A3-6, A12 |
| 4 | erdR (PP_1635) or | LuxR family transcriptional regulator | Deletion, SNP | 5 | A7, A9-12 |
| | mxtR (PP_1695) | or putative Sodium-solute | | | |
| | | symporter/sensory box histidine | | | |
| | | kinase/response regulator | | | |
| 5 | oprD (PP_1206)/ | Basic-amino-acid specific porin | SNP | 4 | A9-12 |
| | PP_1207 | OprD/conserved protein of unknown | | | |
| | | function | | | |
| 6 | infC (PP_2466) | Translation initiation factor IF-3 | In-frame | 3 | A1-2, A6 |
| | | | deletion | | |
| 7 | astA-II (PP_4480) | Arginine N-succinyltransferase, | Early | 3 | A3, A5, A10 |
| | | subunit alpha | termination, | | |
| | | | SNP | | |
| 8 | topA (PP_2139) | DNA topoisomerase I | SNP | 2 | A9 and A10 |
| 9 | PP_2402-calA | 25 Genes^{a} | Deletion, | 2 | A2 and A11 |
| | (PP_2426) | | frameshift | | |
| | | | (PP_2425) | | |
| 10 | PP_3595-PP_3610 | 17 Genes^{a} | Deletion | 2 | A1 and A6 |
| 11 | rpoZ (PP_5301) | DNA-directed RNA polymerase | Frameshift | 2 | A9 and A10 |
| | | subunit omega | | | |

In some embodiments, the microbial strain is a bacterial cell, such as a proteobacteria cell. In some embodiments, the proteobacteria cell is a Gammaproteobacteria cell. In some embodiments, the Gammaproteobacteria cell is a Pseudomonadales or Enterobacterales cell. In some embodiments, the Gammaproteobacteria cell is a Pseudomonadales cell, which is a Pseudomonadaceae cell. In some embodiments, the Pseudomonadaceae cell is a Pseudomonas, Azotobacter, Mesophilobacter, Oblitimonas, Permianibacter, Rugamonas, or Thiopseudomonas cell. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Gammaproteobacteria cell is an Enterobacterales cell, which is an Enterobacteriaceae cell. In some embodiments, the Enterobacteriaceae cell is an Escherichia, Enterobacillus, Enterobacter, Klebsiella, Salmonella, or Shigella cell. In some embodiments, the Escherichia cell is an E. coli, E. albertii, E. fergusonii, E. hermanii, E. marmotae, or E. vulneris. In some embodiments, the host cell is a Gram negative bacterium. In some embodiments, the host cell is a bacterium from the Azotobacter, Escherichia, Salmonella, Vibrio, Pasteurella, Haemophilus, or Pseudomonas genus. In some embodiments, the host cell is a bacterium from the species Escherichia coli, Salmonella enterica, Vibrio cholerae, Pasteurella multocida, Haemophilus influenza, Pseudomonas putida, or Pseudomonas aeruginosa.

In some embodiments, the microbial strain comprises two or more mutations, three or more mutations, four or more mutations, five or more mutations, six or more mutations, seven or more mutations, eight or more mutations, nine or more mutations, or ten or more mutations.

In some embodiments, the microbial strain is capable of growing or dividing in a medium comprising equal to or more than about 4.5 g/L, 5.0 g/L, 5.5 g/L, 6.0 g/L, 6.5 g/L, 7.0 g/L, 7.5 g/L, 8.0 g/L isoprenol, isopentanol, and/or prenol, or a concentration within a range of any two preceding values.

In some embodiments, the microbial strain is genetically modified to produce isoprenol, isopentanol, and/or prenol, or have an increased production of isoprenol, isopentanol, and/or prenol, and optionally any other compound. The genetic modification can be any found in the art.

The present invention provides for a method for producing isoprenol, isopentanol, and/or prenol comprising: (a) providing a genetically modified microbial strain of the present invention, and (b) culturing or growing step the genetically modified microbial strain in a suitable medium to produce isoprenol, isopentanol, and/or prenol.

The present invention provides for a method to identify mutations or generate strains of having the characteristics of the present invention, such as increased tolerance to isoprenol, isopentanol, and/or prenol, or any other compound, comprising one or more of the steps described herein.

Pseudomonas putida KT2440 has been evolved over several generations to generate tolerance to high concentrations of isoprenol. The evolved strains are generated and screened for beneficial mutations and further strain characterization are done. These strains have a significantly higher tolerance to isoprenol (increasing from about 4 g/L to about 8 g/L). This enhanced tolerance facilitates large scale sustainable production of isoprenol from P. putida.

Wildtype and engineered strains of Pseudomonas putida KT2440 are subjected to increasing concentrations of isoprenol from about 4 g/L to about 8 g/L. These strains are evolved through the Tolerance Adaptive Laboratory Evolution (TALE system), and are shown to have significantly enhanced isoprenol tolerance over the parental strains. This is important because high concentrations of isoprenol is toxic to the production chassis P. putida thereby limiting large scale production. With this innovation, one skilled in the art is able to achieve higher titers, rates, and yields in engineered P. putida increasing its commercial viability. This work is novel because the evolved strains acquired mutations that are non-intuitive and have not been reported elsewhere in the context of isoprenol tolerance. Furthermore, it represents the highest tolerance concentration exhibited by this organism to isoprenol. An application of the strain's improved ability to tolerate and grow better on other short chain alcohols such as isopentanol and prenol is being concurrently explored.

In some embodiments, the evolved strains can be further engineered for enhanced production of isoprenol. The strain capabilities have been validated using minimal growth medium, and can be further shown in complex carbon streams, such as plant biomass hydrolysates.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. The workflow and rationale for strain selection for TALE to enhance tolerance to isoprenol in P. putida. (A) Overall workflow for generating and evaluating isoprenol tolerant strains. (B) The proposed isoprenol catabolism pathway in P. putida (Thompson et al., 2020). Genes targeted for deletion to build the starting strains for TALE are denoted in red. (C-E) TALE trajectories of (C) KT2440 WT, (D) IPL300, (E) IPL400. x-axes indicate cumulative cell divisions (CCD). Left and right y-axes indicate growth rates, points (h⁻¹) of evolving populations and isoprenol concentration, solid lines (g/L), respectively. Growth rates were calculated as described in section 2.5 and had high variability due to limited sampling during the evolutions (Materials and Methods).

FIG. 2. Growth rates of the starting strains and evolved isolates in the presence of isoprenol and mutations identified in isolated clones. (A) Maximum specific growth rates (h⁻¹) of the starting strains and the evolved endpoint strains in the presence of 4 g/L and 8 g/L of isoprenol in flasks. (B) Mutated genes identified in the starting, intermediate (gray font) and end-point evolved isolates. Refer to Table 4 for information on the specific type of mutation(s) recovered in each genetic locus.

FIG. 3. Reverse engineering P. putida KT2440 (WT) with commonly mutated targets from a TALE campaign confirmed causality and enabled robust isoprenol production platforms. (A) Relative growth rates of the reverse-engineered strains grown on 4 g/L glucose minimal medium supplemented with 6 g/L isoprenol. (B) Isoprenol production profiles of the reverse engineered strains transformed with the pIY670 plasmid (Banerjee et al., 2024). Transformed strains were grown on glucose (20 g/L) minimal medium with 50 mg/L kanamycin. Cell culture was harvested at 48 h for isoprenol analysis. Values are mean±SD. ⋅ and * indicates p<0.1, p<0.05, respectively.

FIG. 4. Comparative proteomics identified differential protein abundance contributing to improved tolerance in the evolved isolates A10_F53_11 and A12_F63_11. (A) Venn diagrams of the number of differentially expressed proteins vs the mutated genes in the evolved isolates. (B) Significantly upregulated and downregulated proteins in the metabolic pathways and in various subsystems of the evolved isolates of P. putida demonstrating additional mechanisms of isoprenol tolerance. Proteomic profiles of the evolved isolates grown on glucose (4 g/L) minimal medium and supplemented with 4 g/L isoprenol were compared to that of the starting strain IPL400 grown under similar conditions. Significance is defined as log, (Fold change)>±1.5, p<0.05. (C) Growth curves of P. putida strains grown on M9 minimal medium with 4 g/L glucose or 1 g/L isoprenol as carbon source (+IP) in 48 microtiter plates for 24 h. Values are mean±SD of three independent biological replicates. (D) Isoprenol degradation capabilities of WT, the IL400 engineered starting strain and the two evolved isolates when grown on co-substrate glucose. Cells grown on M9 4 g/L glucose and ~1 g/L isoprenol in test tubes were harvested at 24 h and 48 h and the residual isoprenol was measured by GC-FID. Values are mean±SD of three independent biological replicates.

FIG. 5. Rational engineering improved isoprenol titers in the evolved strain A10_F53_I1. (A) Isoprenol titers (mg/L) in the wild type, starting and evolved isolates of P. putida. The strains transformed with the isoprenol production plasmid pIY670 (see Materials and Methods). Cells were harvested at 48 h for analysis. Values are mean±SD. (B) Residual glucose (g/L) measured after 48 h in the cell supernatants. (C) Proteomic analysis of the evolved strain A10_F63_I1 expressing pIY670 compared against the starting strain IPL400 expressing pIY670, harvested at 12 h (Late growth phase), 24 h and 48 h (Production phase). Significantly upregulated and downregulated proteins in various subsystems were plotted as a heatmap. Significance is defined as log, (Fold change)>±1.5, p<0.05. (D) Isoprenol titers (mg/L) in the rationally engineered strains, evolved strains A10_F63_I1 and A12_F54_I1 vs. the starting strain IPL400. Markerless deletion of selected gene targets (fleQ, mvaB, and gacA, see Materials and Methods) identified using proteomics and computational aided metabolic engineering (Banerjee et al., 2024) was carried out. (E) Residual glucose (g/L) measured in the cell supernatants from the production runs harvested at 48 h. Values are mean±SD.

FIG. 6. The overall strategy to generate P. putida KT2440 microbial platforms for isoprenol production. (A) Overall workflow for generating isoprenol-tolerant isolates and demonstration of isoprenol production using them. (B-D) Characteristics of three starting strains, (B) the wildtype KT2440, (C) IPL300, (D) IPL400, and trajectories of the tolerization ALE experiments.

FIG. 7. Growth profiles of the wildtype KT2440 and evolved isolates. (A) Maximum specific growth rates (h₋₁) of the representative evolved strains and the starting strains in the presence of 4 g/L or 8 g/L of isoprenol. (B) Mutated genes identified in the starting and isoprenol-tolerized isolates.

FIG. 8. Relative growth rates of the reverse-engineered strains to investigate the effect of mutated regions. Dot (⋅) and * indicates p<0.1, p<0.05, respectively.

FIG. 9. The evolved strain A5F73 can be rationally engineered for enhancement in isoprenol titers.

FIG. 10. The evolved strain A10F63 shows improved growth in the presence of 4 g/L prenol and isopentanol on glucose minimal medium.

FIG. 11. Construction of the starting strains for tolerization ALE experiments. (A) A scatter plot of gene fitness from RB-TnSeq and gene expression from RNA-Seq when WT cells were grown on isoprenol as a sole carbon source in a minimal M9 condition. Positive values on the x-axis indicate a fitness defect, and the y-axis indicates a fold change of gene expression. A gene fitness for each gene is obtained by dividing the abundance of a gene in cells grown on isoprenol by the abundance in cells grown on glucose. A gene expression fold change was similarly calculated by using DESeq2 (Love et al., 2014) using the glucose condition as a reference. (B) Rb-TnSeq fitness profiles (on various alcohols) of the genes targeted for deletion to build the strains IPL300 and IPL400 for TALE. (A and B) Common genes were colored in blue. (C) Growth of P. putida on M9 minimal medium supplemented with 4 g/L glucose and varying concentrations of isoprenol (0-12 g/L) in a microtiter plate. (D) Growth of WT and engineered strains IPL300 and IPL400 in M9 minimal medium with 4 g/L glucose supplemented with 4 g/L isoprenol grown in flasks. (E) Growth of the wild-type and engineered P. putida KT2440 strains in the M9 medium supplemented in 1 g/L isoprenol as a sole carbon source. (F) Percentage of remaining isoprenol after incubating cells for 48 h in the presence of 150 mg/L isoprenol in M9 minimal medium.

FIG. 12. Common mutations from isoprenol TALE identified in the evolved strains. Mutated regions are colored in yellow. Mutations are highlighted in red as vertical bars.

FIG. 13. Proteomics analysis of selected evolved strains and their isoprenol degradation capabilities. (A) Venn diagram shows the number of common and uniquely up and downregulated proteins in the evolved strains on both glucose minimal media and that supplemented with 4 g/L isoprenol (+IPL). (B) Isoprenol degradation capabilities of WT, the starting strains IPL300 and IPL400 and the evolved strains when grown on co-substrate glucose. Cells grown on M9 4 g/L glucose and ~1 g/L isoprenol in test tubes were harvested at 24 h and 48 h and the residual isoprenol was measured by GC-FID. Values are mean±SD of three independent biological replicates. (C) Differential protein expression of the isoprenol pathway proteins and candidate isoprenol degradation enzymes in the evolved strain A10_F63_I1 expressing pIY670 compared against the parent strain IPL400 expressing pIY670, harvested at 12 h, 24 h and 48 h. Significantly upregulated and downregulated proteins in various subsystems were plotted as a heatmap, Significance is defined as log 2 (Fold change)>±1.5, p<0.05.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “host cell” is used herein to refer to a living biological cell that can be transformed via insertion of an expression vector.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to a host cell or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

Abbreviations used herein: p-CA (or pCA): para-coumarate. GSMM: genome scale metabolic model. ALE: adaptive laboratory evolution. D1a: Design 1a. D1b: Design 1b. SXT, soft X-ray tomography. LAC, linear absorption coefficient. BCD, base catalyzed depolymerization.

The present invention provides for a method for producing isoprenol comprising: (a) providing a genetically modified microbial strain of the present invention, and (b) culturing or growing step the genetically modified microbial strain in a suitable medium to produce isoprenol. In some embodiments, the genetically modified microbial strain produces isoprenol such that the concentration of isoprenol in the medium is equal to or more than about 4.5 g/L, 5.0 g/L, 5.5 g/L, 6.0 g/L, 6.5 g/L, 7.0 g/L, 7.5 g/L, 8.0 g/L isoprenol, or a concentration within a range of any two preceding values. In some embodiments, the method further comprises separating or recovering isoprenol from the medium.

One can modify the expression of a gene encoding any of the enzymes taught herein by a variety of methods in accordance with the methods of the invention. Those skilled in the art would recognize that increasing gene copy number, ribosome binding site strength, promoter strength, and various transcriptional regulators can be employed to alter an enzyme expression level.

### REFERENCES CITED HEREIN

Ackermann, Y. S., Li, W.-J., Op de Hipt, L., Niehoff, P.-J., Casey, W., Polen, T., Köbbing, S.,
Ballerstedt, H., Wynands, B., O'Connor, K., Blank, L. M., Wierckx, N., 2021. Engineering adipic acid metabolism in Pseudomonas putida. Metab. Eng. 67, 29-40.
Ahrné, E., Molzahn, L., Glatter, T., Schmidt, A., 2013. Critical assessment of proteome-wide label-free absolute abundance estimation strategies. Proteomics 13, 2567-2578.
Almqvist, H., Veras, H., Li, K., Garcia Hidalgo, J., Hulteberg, C., Gorwa-Grauslund, M., Skorupa Parachin, N., Carlquist, M., 2021. Muconic Acid Production Using Engineered Pseudomonas putida KT2440 and a Guaiacol-Rich Fraction Derived from Kraft Lignin. ACS Sustain. Chem. Eng.
Babel, H., Krömer, J. O., 2020. Evolutionary engineering of E. coli MG1655 for tolerance against isoprenol. Biotechnol. Biofuels 13, 183.
Banerjee, D., Eng, T., Lau, A. K., Sasaki, Y., Wang, B., Chen, Y., Prahl, J.-P., Singan, V. R., Herbert, R. A., Liu, Y., Tanjore, D., Petzold, C. J., Keasling, J. D., Mukhopadhyay, A., 2020. Genome-scale metabolic rewiring improves titers rates and yields of the non-native product indigoidine at scale. Nat. Commun. 11, 5385.
Banerjee, D., Yunus, I. S., Wang, X., Kim, Jinho, Srinivasan, A., Menchavez, R., Chen, Y., Gin, J. W., Petzold, C. J., Martin, H. G., Magnuson, J. K., Adams, P. D., Simmons, B. A., Mukhopadhyay, A., Kim, Joonhoon, Lee, T. S., 2024. Genome-scale and pathway engineering for the sustainable aviation fuel precursor isoprenol production in Pseudomonas putida. Metab. Eng. 82, 157-170.
Baral, N. R., Yang, M., Harvey, B. G., Simmons, B. A., Mukhopadhyay, A., Lee, T. S., Scown, C. D., 2021. Production Cost and Carbon Footprint of Biomass-Derived Dimethylcyclooctane as a High-Performance Jet Fuel Blendstock. ACS Sustain. Chem. Eng. 9, 11872-11882.
Basler, G., Thompson, M., Tullman-Ercek, D., Keasling, J., 2018. A Pseudomonas putida efflux pump acts on short-chain alcohols. Biotechnol. Biofuels 11, 136.
Belda, E., van Heck, R. G. A., José Lopez-Sanchez, M., Cruveiller, S., Barbe, V., Fraser, C., Klenk, H.-P., Petersen, J., Morgat, A., Nikel, P. I., Vallenet, D., Rouy, Z., Sekowska, A., Martins Dos Santos, V. A. P., de Lorenzo, V., Danchin, A., Médigue, C., 2016. The revisited genome of Pseudomonas putida KT2440 enlightens its value as a robust metabolic chassis. Environ. Microbiol. 18, 3403-3424.
Benavides, P. T., Bartling, A. W., Phillips, S. D., Hawkins, T. R., Singh, A., Zaimes, G. G., Wiatrowski, M., Harris, K., Burli, P. H., Hartley, D., Alleman, T., Fioroni, G., Gaspar, D., 2022. Identification of Key Drivers of Cost and Environmental Impact for Biomass-Derived Fuel for Advanced Multimode Engines Based on Techno-Economic and Life Cycle Analysis. ACS Sustain. Chem. Eng. Webpage for: doi.org/10.1021/acssuschemeng.2c00944.
Bentley, G. J., Narayanan, N., Jha, R. K., Salvachúa, D., Elmore, J. R., Peabody, G. L., Black, B. A., Ramirez, K., De Capite, A., Michener, W. E., Werner, A. Z., Klingeman, D. M., Schindel, H. S., Nelson, R., Foust, L., Guss, A. M., Dale, T., Johnson, C. W., Beckham, G. T., 2020. Engineering glucose metabolism for enhanced muconic acid production in Pseudomonas putida KT2440. Metab. Eng. 59, 64-75.
Borchert, A. J., Bleem, A. C., Lim, H. G., Rychel, K., Dooley, K. D., Kellermyer, Z. A., Hodges, T. L., Palsson, B. O., Beckham, G. T., 2024. Machine learning analysis of RB-TnSeq fitness data predicts functional gene modules in Pseudomonas putida KT2440. mSystems 9, e0094223.
Cha, D., Ha, H. S., Lee, S. K., 2020. Metabolic engineering of Pseudomonas putida for the production of various types of short-chain-length polyhydroxyalkanoates from levulinic acid. Bioresour. Technol. 309, 123332.
Chen, Y., Gin, J., Petzold, C. J., 2022. Discovery proteomic (DIA) LC-MS/MS data acquisition and analysis v2.
Chen, Y., Gin, J. W., Wang, Y., de Raad, M., Tan, S., Hillson, N.J., Northen, T. R., Adams, P. D., Petzold, C. J., 2023. Alkaline-SDS cell lysis of microbes with acetone protein precipitation for proteomic sample preparation in 96-well plate format. PLOS ONE 18, e0288102.
Choe, D., Szubin, R., Poudel, S., Sastry, A., Song, Y., Lee, Y., Cho, S., Palsson, B., Cho, B.-K., 2021. RiboRid: A low cost, advanced, and ultra-efficient method to remove ribosomal RNA for bacterial transcriptomics. PLOS Genet. 17, e1009821.
Czajka, J. J., Banerjee, D., Eng, T., Menasalvas, J., Yan, C., Munoz, N. M., Poirier, B. C., Kim,
Y.-M., Baker, S. E., Tang, Y. J., Mukhopadhyay, A., 2022. Tuning a high performing multiplexed-CRISPRi Pseudomonas putida strain to further enhance indigoidine production. Metab. Eng. Commun. 15, e00206.
Deatherage, D. E., Barrick, J. E., 2014. Identification of mutations in laboratory-evolved microbes from next-generation sequencing data using breseq. Methods Mol. Biol. 1151, 165-188.
del Castillo, T., Duque, E., Ramos, J. L., 2008. A set of activators and repressors control peripheral glucose pathways in Pseudomonas putida to yield a common central intermediate. J. Bacteriol. 190, 2331-2339.
Dyga, M., Keller, A., Hasse, H., 2021. Vapor-liquid equilibria and chemical equilibria in the system (formaldehyde+water+isoprenol). Ind. Eng. Chem Res. 60, 4471-4483.
Elmore, J. R., Dexter, G. N., Salvachua, D., O'Brien, M., Klingeman, D. M., Gorday, K., Michener, J. K., Peterson, D. J., Beckham, G. T., Guss, A. M., 2020. Engineered Pseudomonas putida simultaneously catabolizes five major components of corn stover lignocellulose: Glucose, xylose, arabinose, p-coumaric acid, and acetic acid. Metab. Eng. 62, 62-71.
Emelianov, G., Song, D.-U., Jang, N., Ko, M., Kim, S. K., Rha, E., Shin, J., Kwon, K. K., Kim, H., Lee, D.-H., Lee, H., Lee, S.-G., 2024. Engineered Methylococcus capsulatus Bath for efficient methane conversion to isoprene. Bioresour. Technol. 393, 130098.
Eng, T., Banerjee, D., Menasalvas, J., Chen, Y., Gin, J., Choudhary, H., Baidoo, E., Chen, J. H., Ekman, A., Kakumanu, R., Diercks, Y. L., Codik, A., Larabell, C., Gladden, J., Simmons, B. A., Keasling, J. D., Petzold, C. J., Mukhopadhyay, A., 2023. Maximizing microbial bioproduction from sustainable carbon sources using iterative systems engineering. Cell Rep. 42, 113087.
Fong, S. S., Nanchen, A., Palsson, B. O., Sauer, U., 2006. Latent pathway activation and increased pathway capacity enable Escherichia coli adaptation to loss of key metabolic enzymes. J. Biol. Chem. 281, 8024-8033.
Gauttam, R., Eng, T., Zhao, Z., Ul Ain Rana, Q., Simmons, B. A., Yoshikuni, Y., Mukhopadhyay, A., Singer, S. W., 2023. Development of genetic tools for heterologous protein expression in a pentose-utilizing environmental isolate of Pseudomonas putida. Microb. Biotechnol. 16, 645-661.
Goodarzi, H., Bennett, B. D., Amini, S., Reaves, M. L., Hottes, A. K., Rabinowitz, J. D., Tavazoie, S., 2010. Regulatory and metabolic rewiring during laboratory evolution of ethanol tolerance in E. coli. Mol. Syst. Biol. 6, 378.
Halle, L., Hollmann, N., Tenhaef, N., Mbengi, L., Glitz, C., Wiechert, W., Polen, T., Baumgart, M., Bott, M., Noack, S., 2023. Robotic workflows for automated long-term adaptive laboratory evolution: improving ethanol utilization by Corynebacterium glutamicum. Microb. Cell Fact. 22, 175.
Hernandez-Arranz, S., Perez-Gil, J., Marshall-Sabey, D., Rodriguez-Concepcion, M., 2019. Engineering Pseudomonas putida for isoprenoid production by manipulating endogenous and shunt pathways supplying precursors. Microb. Cell Fact. 18, 152.
Huffer, S., Clark, M. E., Ning, J. C., Blanch, H. W., Clark, D. S., 2011. Role of alcohols in growth, lipid composition, and membrane fluidity of yeasts, bacteria, and archaea. Appl. Environ. Microbiol. 77, 6400-6408.
Iwasaki, K., Uchiyama, H., Yagi, O., Kurabayashi, T., Ishizuka, K., Takamura, Y., 1994. Transformation ofPseudomonas putida by Electroporation. Biosci. Biotechnol. Biochem. 58, 851-854.
Kang, M., Kim, K., Choe, D., Cho, S., Kim, S. C., Palsson, B., Cho, B.-K., 2019. Inactivation of a Mismatch-Repair System Diversifies Genotypic Landscape of Escherichia coli During Adaptive Laboratory Evolution. Front. Microbiol. 10, 1845.
Kant, G., Pandey, A., Shekhar, H., Srivastava, S., 2023. Enhanced bio-synthesis of isoprene via modifying mevalonate and methylerythritol phosphate pathways for industrial application: A review. Process Biochemistry 130, 256-271.
Karp, P. D., Billington, R., Caspi, R., Fulcher, C. A., Latendresse, M., Kothari, A., Keseler, I. M., Krummenacker, M., Midford, P. E., Ong, Q., Ong, W. K., Paley, S. M., Subhraveti, P., 2019. The BioCyc collection of microbial genomes and metabolic pathways. Brief. Bioinformatics 20, 1085-1093.
Kim, Junyoung, Lee, S., Darlington, A. P. S., Kim, Juhyun, 2024. Impact of fleQ Deficiency on Resource Allocation and Heterologous Gene Expression in Pseudomonas putida Across Various Growth Media. Microb. Biotechnol. 17, e70054.
LaCroix, R. A., Sandberg, T. E., O'Brien, E. J., Utrilla, J., Ebrahim, A., Guzman, G. I., Szubin, R., Palsson, B. O., Feist, A. M., 2015. Use of adaptive laboratory evolution to discover key mutations enabling rapid growth of Escherichia coli K-12 MG1655 on glucose minimal medium. Appl. Environ. Microbiol. 81, 17-30.
Langmead, B., Salzberg, S. L., 2012. Fast gapped-read alignment with Bowtie 2. Nat. Methods 9, 357-359 . . .
Lawrence, M., Huber, W., Pages, H., Aboyoun, P., Carlson, M., Gentleman, R., Morgan, M. T., Carey, V. J., 2013. Software for computing and annotating genomic ranges. PLOS Comput. Biol. 9, e1003118.
Lennen, R. M., Lim, H. G., Jensen, K., Mohammed, E. T., Phaneuf, P. V., Noh, M. H., Malla, S., Börner, R. A., Chekina, K., Özdemir, E., Bonde, I., Koza, A., Maury, J., Pedersen, L. E., Schöning, L. Y., Sonnenschein, N., Palsson, B. O., Nielsen, A. T., Sommer, M. O. A., Herrgård, M. J., Feist, A. M., 2023. Laboratory evolution reveals general and specific tolerance mechanisms for commodity chemicals. Metab. Eng. 76, 179-192.
Lim, H. G., Eng, T., Banerjee, D., Alarcon, G., Lau, A. K., Park, M.-R., Simmons, B. A., Palsson, B. O., Singer, S. W., Mukhopadhyay, A., Feist, A. M., 2021. Generation of Pseudomonas putida KT2440 Strains with Efficient Utilization of Xylose and Galactose via Adaptive Laboratory Evolution. ACS Sustain. Chem. Eng. 9, 11512-11523.
Lim, H. G., Fong, B., Alarcon, G., Magurudeniya, H. D., Eng, T., Szubin, R., Olson, C. A., Palsson, B. O., Gladden, J. M., Simmons, B. A., Mukhopadhyay, A., Singer, S. W., Feist, A. M., 2020. Generation of ionic liquid tolerant Pseudomonas putida KT2440 strains via adaptive laboratory evolution. Green Chem. 22, 5677-5690.
Lim, H. G., Rychel, K., Sastry, A. V., Bentley, G. J., Mueller, J., Schindel, H. S., Larsen, P. E., Laible, P. D., Guss, A. M., Niu, W., Johnson, C. W., Beckham, G. T., Feist, A. M., Palsson, B. O., 2022. Machine-learning from Pseudomonas putida KT2440 transcriptomes reveals its transcriptional regulatory network. Metab. Eng. 72, 297-310.
Ling, C., Peabody, G. L., Salvachúa, D., Kim, Y.-M., Kneucker, C. M., Calvey, C. H., Monninger, M. A., Munoz, N. M., Poirier, B. C., Ramirez, K. J., St John, P. C., Woodworth, S. P., Magnuson, J. K., Burnum-Johnson, K. E., Guss, A. M., Johnson, C. W., Beckham, G. T., 2022. Muconic acid production from glucose and xylose in Pseudomonas putida via evolution and metabolic engineering. Nat. Commun. 13, 4925.
Linger, J. G., Vardon, D. R., Guarnieri, M. T., Karp, E. M., Hunsinger, G. B., Franden, M. A., Johnson, C. W., Chupka, G., Strathmann, T. J., Pienkos, P. T., Beckham, G. T., 2014. Lignin valorization through integrated biological funneling and chemical catalysis. Proc Natl Acad Sci USA 111, 12013-12018.
Love, M. I., Huber, W., Anders, S., 2014. Moderated estimation of fold change and dispersion for RNA-seq data with DESeq2. Genome Biol. 15, 550.
Martin-Pascual, M., Batianis, C., Bruinsma, L., Asin-Garcia, E., Garcia-Morales, L., Weusthuis, R. A., van Kranenburg, R., Martins Dos Santos, V. A. P., 2021. A navigation guide of synthetic biology tools for Pseudomonas putida. Biotechnol. Adv. 49, 107732.
Mavrommati, M., Papanikolaou, S., Aggelis, G., 2023. Improving ethanol tolerance of Saccharomyces cerevisiae through adaptive laboratory evolution using high ethanol concentrations as a selective pressure. Process Biochemistry 124, 280-289.
Mohamed, E. T., Wang, S., Lennen, R. M., Herrgård, M. J., Simmons, B. A., Singer, S. W., Feist, A. M., 2017. Generation of a platform strain for ionic liquid tolerance using adaptive laboratory evolution. Microb. Cell Fact. 16, 204.
Mohamed, E. T., Werner, A. Z., Salvachúa, D., Singer, C. A., Szostkiewicz, K., Rafael Jiménez-Díaz, M., Eng, T., Radi, M. S., Simmons, B. A., Mukhopadhyay, A., Herrgård, M. J., Singer, S. W., Beckham, G. T., Feist, A. M., 2020. Adaptive laboratory evolution of Pseudomonas putida KT2440 improves p-coumaric and ferulic acid catabolismand tolerance. Metab. Eng. Commun. 11, e00143.
Mukhopadhyay, A., 2015. Tolerance engineering in bacteria for the production of advanced biofuels and chemicals. Trends Microbiol. 23, 498-508.
Mundhada, H., Seoane, J. M., Schneider, K., Koza, A., Christensen, H. B., Klein, T., Phaneuf, P. V., Herrgard, M., Feist, A. M., Nielsen, A. T., 2017. Increased production of L-serine in Escherichia coli through Adaptive Laboratory Evolution. Metab. Eng. 39, 141-150.
Nguyen-Vo, T. P., Liang, Y., Sankaranarayanan, M., Seol, E., Chun, A. Y., Ashok, S., Chauhan, A. S., Kim, J. R., Park, S., 2019. Development of 3-hydroxypropionic-acid-tolerant strain of Escherichia coli W and role of minor global regulator yieP. Metab. Eng. 53, 48-58.
Nikel, P. I., de Lorenzo, V., 2018. Pseudomonas putida as a functional chassis for industrial biocatalysis: From native biochemistry to trans-metabolism. Metab. Eng. 50, 142-155.
Nikel, P. I., Martínez-García, E., de Lorenzo, V., 2014. Biotechnological domestication of pseudomonads using synthetic biology. Nat. Rev. Microbiol. 12, 368-379.
Niu, W., Willett, H., Mueller, J., He, X., Kramer, L., Ma, B., Guo, J., 2020. Direct biosynthesis of adipic acid from lignin-derived aromatics using engineered Pseudomonas putida KT2440. Metab. Eng. 59, 151-161.
Pang, B., Li, J., Eiben, C. B., Oksen, E., Barcelos, C., Chen, R., Englund, E., Sundstrom, E., Keasling, J. D., 2021. Lepidopteran mevalonate pathway optimization in Escherichia coli efficiently produces isoprenol analogs for next-generation biofuels. Metab. Eng. 68, 210-219.
Perez-Riverol, Y., Bai, J., Bandla, C., García-Seisdedos, D., Hewapathirana, S., Kamatchinathan, S., Kundu, D. J., Prakash, A., Frericks-Zipper, A., Eisenacher, M., Walzer, M., Wang, S., Brazma, A., Vizcaíno, J. A., 2022. The PRIDE database resources in 2022: a hub for mass spectrometry-based proteomics evidences. Nucleic Acids Res. 50, D543-D552.
Phaneuf, P. V., Gosting, D., Palsson, B. O., Feist, A. M., 2019. ALEdb 1.0: a database of mutations from adaptive laboratory evolution experimentation. Nucleic Acids Res. 47, D1164-D1171.
Romero-Steiner, S., Parales, R. E., Harwood, C. S., Houghton, J. E., 1994. Characterization of the pcaR regulatory gene from Pseudomonas putida, which is required for the complete degradation of p-hydroxybenzoate. J. Bacteriol. 176, 5771-5779.
Rosenkoetter, K. E., Kennedy, C. R., Chirik, P. J., Harvey, B. G., 2019. [4+4]-cycloaddition of isoprene for the production of high-performance bio-based jet fuel. Green Chem. 21, 5616-5623.
Sandberg, T. E., Lloyd, C. J., Palsson, B. O., Feist, A. M., 2017. Laboratory evolution to alternating substrate environments yields distinct phenotypic and genetic adaptive strategies. Appl. Environ. Microbiol. 83, e00410-17.
Silva, J. C., Gorenstein, M. V., Li, G.-Z., Vissers, J. P. C., Geromanos, S. J., 2006. Absolute quantification of proteins by LCMSE: a virtue of parallel MS acquisition. Mol. Cell. Proteomics 5, 144-156.
Thompson, M. G., Incha, M. R., Pearson, A. N., Schmidt, M., Sharpless, W. A., Eiben, C. B., Cruz-Morales, P., Blake-Hedges, J. M., Liu, Y., Adams, C. A., Haushalter, R. W., Krishna, R. N., Lichtner, P., Blank, L. M., Mukhopadhyay, A., Deutschbauer, A. M., Shih, P. M., Keasling, J. D., 2020. Fatty acid and alcohol metabolism in Pseudomonas putida: functional analysis using random barcode transposon sequencing. Appl. Environ. Microbiol. 86, e01665-20.
Valencia, L. E., Incha, M. R., Schmidt, M., Pearson, A. N., Thompson, M. G., Roberts, J. B., Mehling, M., Yin, K., Sun, N., Oka, A., Shih, P. M., Blank, L. M., Gladden, J., Keasling, J. D., 2022. Engineering Pseudomonas putida KT2440 for chain length tailored free fatty acid and oleochemical production. Commun. Biol. 5, 1363.
Van der Auwera, G. A., Carneiro, M. O., Hartl, C., Poplin, R., Del Angel, G., Levy-Moonshine, A., Jordan, T., Shakir, K., Roazen, D., Thibault, J., Banks, E., Garimella, K. V., Altshuler, D., Gabriel, S., DePristo, M. A., 2013. From FastQ data to high confidence variant calls: the Genome Analysis Toolkit best practices pipeline. Curr. Protoc. Bioinformatics 11, 11.10.1-11.10.33.
Van der Auwera, G. A., O'Connor, B. D., 2020. Genomics in the Cloud: Using Docker, GATK, and WDL in Terra. O'Reilly Media.
van Duuren, J. B. J. H., Wijte, D., Leprince, A., Karge, B., Puchałka, J., Wery, J., Dos Santos, V. A. P. M., Eggink, G., Mars, A. E., 2011. Generation of a catR deficient mutant of P. putida KT2440 that produces cis, cis-muconate from benzoate at high rate and yield. J. Biotechnol. 156, 163-172.
Wada, A., Prates, É. T., Hirano, R., Werner, A. Z., Kamimura, N., Jacobson, D. A., Beckham, G. T., Masai, E., 2021. Characterization of aromatic acid/proton symporters in Pseudomonas putida KT2440 toward efficient microbial conversion of lignin-related aromatics. Metab. Eng. 64, 167-179.
Wang, X., Baidoo, E. E. K., Kakumanu, R., Xie, S., Mukhopadhyay, A., Lee, T. S., 2022. Engineering isoprenoids production in metabolically versatile microbial host Pseudomonas putida. Biotechnol. Biofuels Bioprod. 15, 137.
Wannier, T. M., Nyerges, A., Kuchwara, H. M., Czikkely, M., Balogh, D., Filsinger, G. T., Borders, N.C., Gregg, C. J., Lajoie, M. J., Rios, X., Pál, C., Church, G. M., 2020. Improved bacterial recombineering by parallelized protein discovery. Proc Natl Acad Sci USA 117, 13689-13698.
Weinel, C., Nelson, K. E., Tümmler, B., 2002. Global features of the Pseudomonas putida KT2440 genome sequence. Environ. Microbiol. 4, 809-818.
Zheng, Y., Liu, Q., Li, L., Qin, W., Yang, J., Zhang, H., Jiang, X., Cheng, T., Liu, W., Xu, X., Xian, M., 2013. Metabolic engineering of Escherichia coli for high-specificity production of isoprenol and prenol as next generation of biofuels. Biotechnol. Biofuels 6, 57.
Zou, L., Ouyang, S., Hu, Y., Zheng, Z., Ouyang, J., 2021. Efficient lactic acid production from dilute acid-pretreated lignocellulosic biomass by a synthetic consortium of engineered Pseudomonas putida and Bacillus coagulans. Biotechnol. Biofuels 14, 227.
Kang A, George K W, Wang G, Baidoo E, Keasling J D, Lee T S. Isopentenyl diphosphate (IPP)-bypass mevalonate pathways for isopentenol production. Metab Eng. 2016 March; 34:25-35.
Sasaki Y, Eng T, Herbert R A, Trinh J, Chen Y, Rodriguez A, et al. Engineering Corynebacterium glutamicum to produce the biogasoline isopentenol from plant biomass hydrolysates. Biotechnol Biofuels. 2019 Feb. 27; 12:41.
LaCroix, R. A et al. Appl. Environ. Microbiol. 83:1-14. (2017).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Evolution Guided Tolerance Engineering of Pseudomonas putida KT2440 for Production of the Sustainable Aviation Fuel Precursor Isoprenol

Isoprenol (3-methyl-3-buten-1-ol) is a sustainable aviation fuel precursor and commodity chemical which can be biomanufactured microbially from renewable carbon streams. Its production has been demonstrated in Pseudomonas putida KT2440 but its titers, rates, and yields have yet to reach commercially viable levels, potentially due to its inherent toxicity. We hypothesized that utilization of Tolerization Adaptive Laboratory Evolution (TALE) would generate P. putida hosts more tolerant to isoprenol and suitable for enhanced production phenotypes. Here, we performed a comprehensive TALE campaign using three strains, the wild-type and two strains lacking subsets of known isoprenol catabolism and transport functions in quadruplicate independently evolved lineages. Several evolved clones from each starting strain displayed robust growth (up to 0.2 h⁻¹) at 8 g/L of isoprenol, where starting strains could not grow. Whole genome resequencing of the 12 independent strain lineages identified convergent mutations. Reverse engineering four commonly mutated regions individually (gnuR, ttgB-PP_1394, PP_3024-PP_5558, PP_1695) each resulted in a partial recovery of the tolerance phenotypes observed in the evolved strains. Additionally, a proteomics-guided deletion of the master motility regulator, fleQ, in an evolved clone alleviated the tolerance vs. production trade-off, restoring isoprenol titers and consumption to levels observed in the starting strains. Collectively, this work demonstrated that an integrated strategy of laboratory evolution and rational engineering was effective to develop robust biofuel production hosts with minimized product toxicity.

In this study, we performed an in-depth comparative TALE experiment to improve and understand mechanisms of tolerance of P. putida KT2440 (hereafter, KT2440) to isoprenol. In addition to the wildtype KT2440 strain (WT), we constructed two additional starting strains lacking isoprenol catabolism and a putative isoprenol efflux pump to generate tolerized strains via the known routes. All three strains were tolerized to gradually increasing concentrations of isoprenol. We successfully obtained evolved isolates that can robustly grow at 8 g/L isoprenol, a lethal level to WT (despite its ability to catabolize isoprenol) and characterized the resulting systems-level changes by DNA resequencing and shotgun proteomics. While isoprenol production was initially low in evolved clones, rational engineering approaches based on proteomics analysis restored isoprenol titers to comparable levels to the starting control strain.

### Materials and Methods

### Bacterial Cells, Plasmids, and Reagents

Bacterial strains, plasmids and oligonucleotides used in this study were listed in Tables 2 and 3. Oligonucleotides were synthesized by Integrated DNA Technologies (IDT, Coralville, IA, USA). Plasmid cloning was performed by using a NEBuilder HiFi DNA Assembly Master Mix from New England Biolabs (NEB, Ipswich, MA, USA). Q5 and HotStartTaq polymerases were also purchased from NEB. Scar-less gene deletion was performed by following the conjugation protocol using E. coli S-17 as previously described (Lim et al., 2020). Alternatively, we used recombineering to generate deletion mutants with oligonucleotides in conjunction with a Cpf1/RecT two-plasmid system (Czajka et al., 2022; Wannier et al., 2020). All chemicals were purchased from Sigma-Aldrich (St. Louis, MO, USA) unless otherwise mentioned. We note that commercial isoprenol stock (97%) contains formaldehyde as an impurity (Babel and Krömer, 2020; Dyga et al., 2021).

### Bacterial Cultivation

All bacterial strains were routinely cultivated on LB medium or the modified M9 minimal medium. The M9 medium contained 2 g/L (NH₄)₂SO₄, 6.8 g/L Na₂HPO₄, 3 g/L KH₂PO₄, 0.5 g/L NaCl, 2 mM MgSO₄, 0.1 mM CaCl₂), 500 μL/L 2000× trace element solution (Lim et al., 2020; Linger et al., 2014). As a carbon source, 4 g/L glucose was added to the minimal medium unless otherwise stated. To monitor cell growth, colonies grown in LB agar plates were picked and inoculated into 15 mL of the M9 medium contained in 30 mL cylindrical flasks. After overnight growth, cultures were re-inoculated at an OD₆₀₀ (optical density at 600 nm) of 0.05. Once OD₆₀₀ reached 0.6-0.8, cells were further inoculated at OD₆₀₀ of 0.05 as main cultures. For small scale cultures (i.e., microtiter plates), subsequent cultures were inoculated at OD₆₀₀ of 0.1 in 200 μL of a culture medium. Maximum growth rates were calculated by plotting slope of ln(OD₆₀₀) vs. time during exponential growth.

TALE was performed by passaging bacterial cultures to 15 mL of fresh medium in flasks once or twice a day at an initial OD₆₀₀ value of 0.05. Cell growth was monitored by measuring OD₆₀₀ of cultures over time. Concentrations of isoprenol were increased from 4 g/L (46 mM) by a step size of 0.5 or 1 g/L. When an increase in isoprenol concentration resulted in complete growth inhibition, the previous culture was passaged in non-selective M9 medium and the experiment was restarted with the prior isoprenol concentration. Each evolved isolate was named according to the ALEdb convention (Phaneuf et al., 2019) that uses the ALE experiment number (A), flask number (F), and isolate number (I).

### Genome and Transcriptome Sequencing

For whole-genome sequencing, genomic DNA was extracted from 500 μL of saturated cell cultures by using a Mag-Bind® Bacterial DNA 96 Kit from Omega Bio-tek (Norcross, GA, USA) and KingFisher™ Duo Prime Purification System from Thermo Fisher Scientific (Waltham, MA, USA). Genomic sequencing libraries were prepared by using a plex Well™ 96 kit from seqWell (Beverly, MA, USA). Raw files were processed by an in-lab analysis pipeline that utilizes Breseq (version 0.33.1) (Deatherage and Barrick, 2014), GATK (Van der Auwera and O'Connor, 2020; Van der Auwera et al., 2013), and Bowtie2 (Langmead and Salzberg, 2012) (version 2.3.4.1). Mutation analysis results were uploaded to ALEdb v1.0 (Phaneuf et al., 2019) and accessible at the webpage for: aledb.org/with “Pputida_isoprenol_TALE” as the project name. Sequencing coverage varied between 10-50×; the coverages of endpoint clones were at least 30×. Starting strain mutations were filtered out. Raw-read files were deposited to NCBI SRA with a BioProject number of PRJNA1187681.

For transcriptome sequencing, the log phase WT cells grown on either glucose or isoprenol as a sole carbon source were taken. The total RNA was extracted from 3 mL of cell cultures at the exponential growth phase (OD₆₀₀ 0.6-0.8), stabilized by mixing 6 mL of an RNAprotect Bacteria Reagent from Qiagen (Hilden, Germany). Total RNA was extracted by using a Quick-RNA Fungal/Bacterial Microprep Kit from Zymo (Irvine, CA). Subsequently, ribosomal RNA was depleted by following the RiboRid (Choe et al., 2021) and OligoRid protocol using anti-ribosomal RNA oligonucleotides designed for KT2440 (Lim et al., 2020). Transcriptome sequencing libraries were prepared by using either a Swift Rapid RNA Library Kit from Swift (Ann Arbor, MI) or KAPA RNA HyperPrep Kit from Roche Sequencing (Pleasanton, CA). Prepared libraries were sequenced by using an illumina (San Diego, CA) NextSeq or NovaSeq platform. Raw reads were analyzed by using Bowtie2, DESeq2 (Love et al., 2014), summarizeOverlaps (Lawrence et al., 2013). Raw read files were deposited at Gene Expression Omnibus with an accession number of GSE281392.

### Biomass Quantification

Biomass was determined by monitoring absorbance at 600 nm (OD₆₀₀) by using an Infinite 200 PRO microtiter plate reader from Tecan (Männedorf, Switzerland), BioTek Synergy microplate reader (Agilent Technologies, USA), or a Biomate 3S benchtop spectrophotometer from Thermo Fisher Scientific (Waltham, MA, USA) wherever appropriate. Microtiter plate OD₆₀₀ readings measured by the Infinite 200 Pro microtiter plate reader were multiplied by a factor of 0.2211 for conversion into readings by the bench-top spectrophotometer.

### Shotgun Proteomic Analysis

Cultures at the exponential growth phase were harvested by centrifugation and stored at −80° C. until analysis. After all samples were collected, protein was extracted from the pellets and tryptic peptides were prepared by following established proteomic sample preparation procedures (Chen et al., 2023). Briefly, cell pellets were resuspended in Qiagen P2 Lysis Buffer (Qiagen Sciences, Germantown, MD, Cat. #19052) for cell lysis. Proteins were precipitated with addition of 1 mM NaCl and 4× volume acetone, followed by two additional washes with 80% acetone in water. The recovered protein pellet was homogenized by pipetting mixing with 100 mM ammonium bicarbonate in 20% methanol. Protein concentration was determined by the DC protein assay (Bio-Rad Inc, Hercules, CA). Protein reduction was accomplished using 5 mM Tris-2-(carboxyethyl) phosphine for 30 min at room temperature, and alkylation was performed with 10 mM iodoacetamide as a final concentration for 30 min at room temperature in the dark. Overnight digestion with trypsin was accomplished with a 1:50 trypsin:total protein ratio. The resulting peptide samples were analyzed on an Agilent 1290 UHPLC system coupled to a Thermo Scientific Orbitrap Exploris 480 mass spectrometer for discovery proteomics (Chen et al., 2022). Briefly, peptides were loaded onto an Ascentis ES-C18 Column (Sigma-Aldrich, St. Louis, MO) and separated with a 10 min LC gradient starting at 98% solvent A (0.1% FA in H2O) and 2% solvent B (0.1% FA in ACN) to 65% solvent A and 35% solvent B. Eluting peptides were introduced to the mass spectrometer operating in positive-ion mode and were measured in data-independent acquisition (DIA) mode with a duty cycle of 3 survey scans from m/z 380 to m/z 985 and 45 MS2 scans with precursor isolation width of 13.5 m/z to cover the mass range. DIA raw data files were analyzed by an integrated software suite DIA-NN. The database used in the DIA-NN search (library-free mode) is the latest Uniprot P. putida KT2440 proteome FASTA sequence plus the protein sequences of heterogeneous pathway genes and common proteomic contaminants. DIA-NN determines mass tolerances automatically based on first pass analysis of the samples with automated determination of optimal mass accuracies. The retention time extraction window was determined individually for all MS runs analyzed via the automated optimization procedure implemented in DIA-NN. Protein inference was enabled, and the quantification strategy was set to Robust LC=High Accuracy. Output main DIA-NN reports were filtered with a global FDR=0.01 on both the precursor level and protein group level. The Top3 method, which is the average MS signal response of the three most intense tryptic peptides of each identified protein, was used to plot the quantity of the targeted proteins in the samples (Ahrné et al., 2013; Silva et al., 2006). With this method, an average of 2,375 out of 5,565 putative proteins were quantified.

The generated mass spectrometry proteomics data have been deposited to the ProteomeXchange Consortium via the PRIDE partner repository with the dataset identifier PXD054609 (Perez-Riverol et al., 2022). DIA-NN is freely available for download from the webpage for: github.com/vdemichev/DiaNN.

### Isoprenol Production and Metabolite Quantification

For isoprenol production, P. putida strains were transformed with approximately 100 ng purified plasmid pIY670 (Banerjee et al., 2024) via electroporation (Iwasaki et al., 1994) and selected on solid LB agar containing 50 μg/mL kanamycin. Plasmid pIY670 was prepared for electroporation by extraction from E. coli XL-1 using standard alkaline lysis and silica column purification (Qiagen Inc, Plasmid DNA Miniprep Kit). After transformation, a single well formed P. putida colony was picked from the agar plate and used to inoculate a 5 mL LB culture in a test tube which was continuously shaken at 200 rpm and maintained at 30° C. Cultures were adapted twice in NREL M9 minimal medium (Linger et al., 2014) by back dilution roughly every 24 hours, aiming for robust cell growth. At the third back-dilution, cells were inoculated to a starting OD₆₀₀ of 0.1 in 20-mL test tubes containing 5 mL NREL M9 minimal medium as described in Section 2.2 xxxxxxxxxx with 20 g/L glucose as carbon source and 50 μg/mL kanamycin. The isoprenol pathway was induced by adding arabinose at 2 g/L at T=0 h. 200 μL of the culture was harvested at 12 h, 24 h and 48 h for analysis of isoprenol, residual glucose and proteomics respectively. Extraction and analysis of isoprenol was carried out as described in a previous study (Banerjee et al., 2024). Briefly, whole cells were extracted with equal volumes of ethyl acetate, vortexed for 15 min and centrifuged at 14,000×g for 3 min. 100 μL of the upper organic phase was analyzed using gas chromatography connected to a flame ionization detector (GC-FID) with the following settings-column: 15 m×0.32 mm×0.5 μm polar DB-Wax column (Agilent Technologies, USA), carrier gas: helium at a flow rate of 2.22 mL/min; flame: hydrogen; oven temperature gradient: initial hold at 40° C. for 1 min, ramped up at the rate of 15° C./min to 100° C. and finally heated to to 230° C. and held at this temperature for 3 min. Isoprenol was quantified from a standard calibration curve using an authentic standard (Sigma Aldrich, USA). Residual glucose and organic acids (lactate, acetate and succinate) were measured using a Agilent 1260 HPLC (Agilent Technologies, USA) equipped with an Aminex HPX-87H column with 4 mM sulfuric acid as the mobile phase at 0.6 mL min⁻¹ and analyzed using a refractive index detector (RID). Standard calibration curves were generated using serial dilutions of the stocks of the respective authentic analytes. Samples were analyzed as three independent biological replicates.

### Results and Discussion

Design of an Isoprenol TALE Campaign with Different Starting Strains Based on RB-TnSeq and Transcriptome Analysis

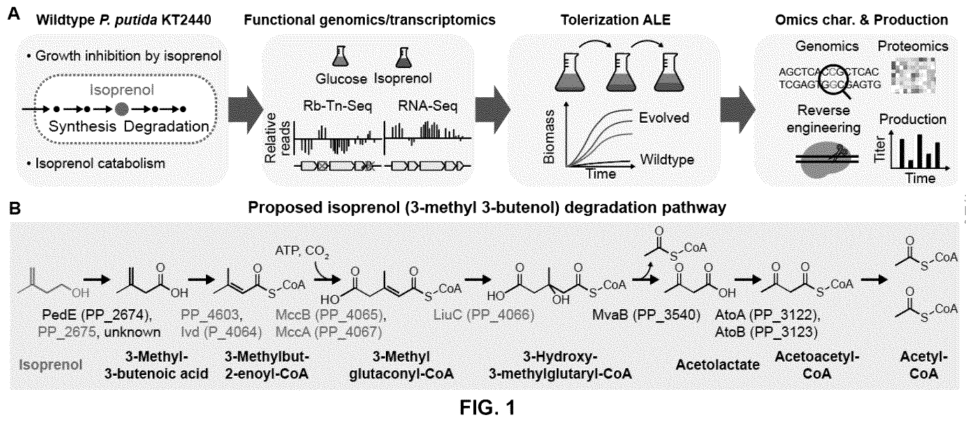

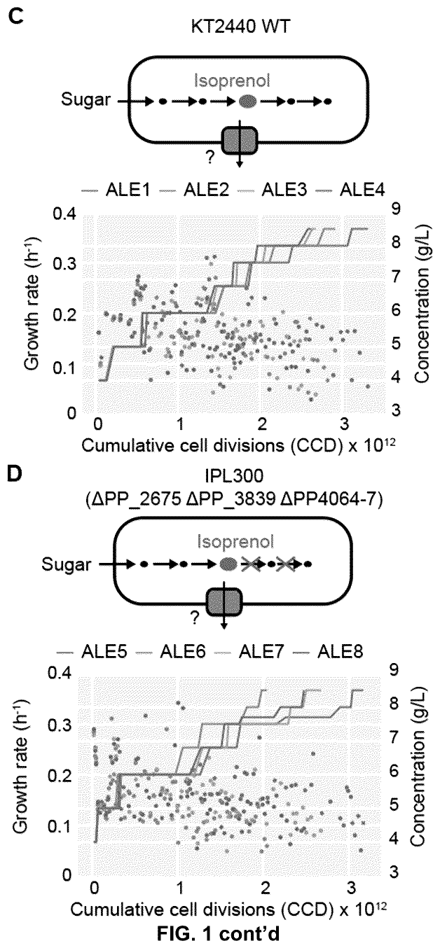

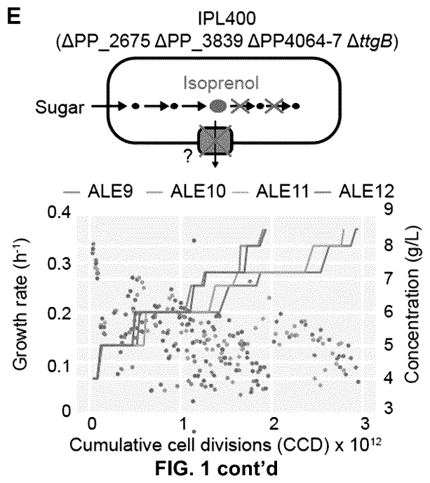

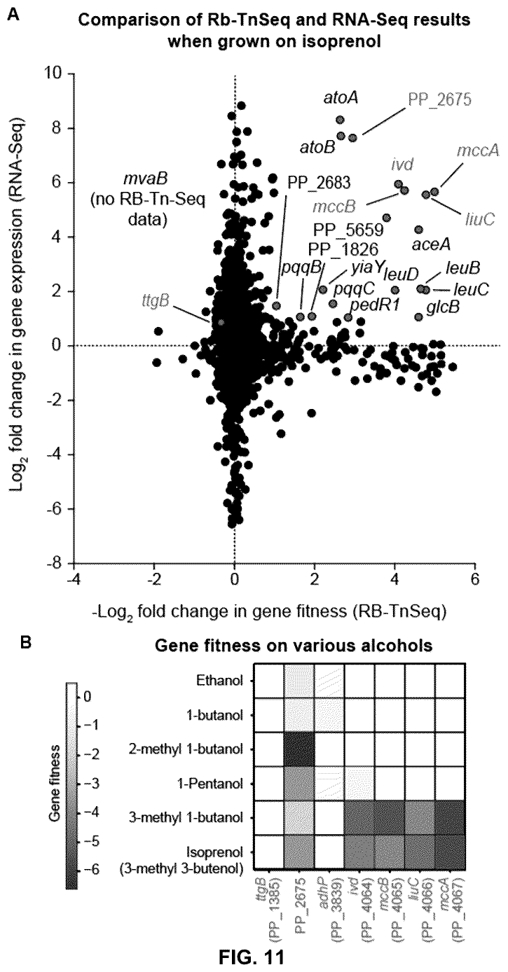

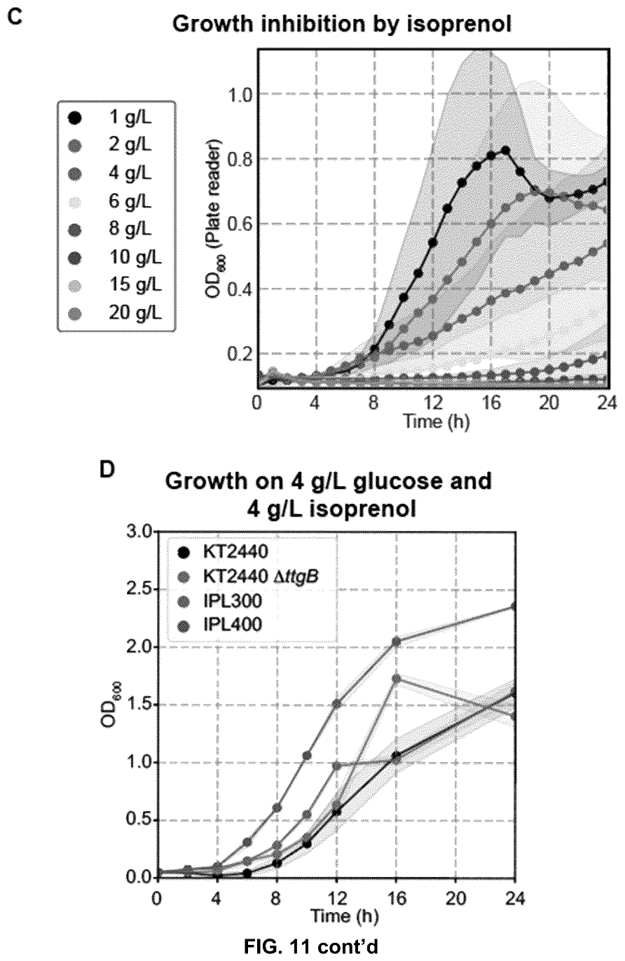

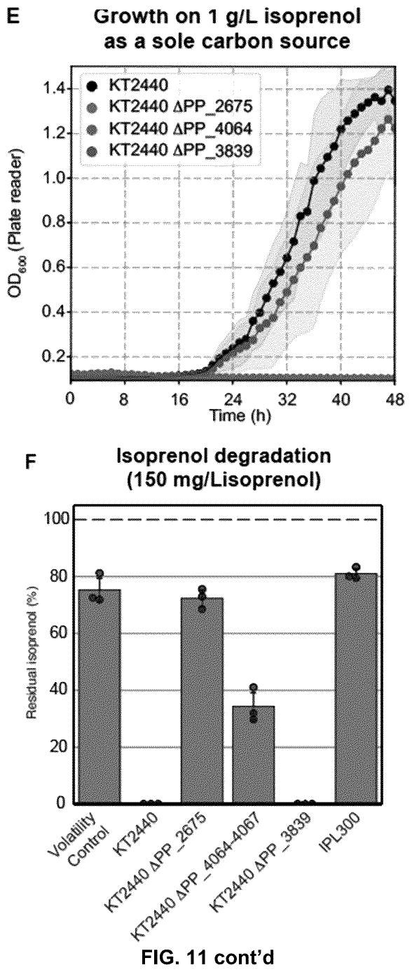

RB-Tn-Seq and RNA-Seq analyses were utilized to identify pathways and transporters related to isoprenol metabolism in P. putida and to generate multiple starting strains for the TALE campaign (FIG. 1, panel A-C). We hypothesized that KT2440 employed two potential mechanisms to mitigate isoprenol toxicity: (1) utilization of isoprenol as a carbon source through catabolism, and (2) secretion of isoprenol from the cytoplasm using a transporter, based on protein homology with a probable isoprenol efflux pump, T1E-0242 (TtgB), from P. putida DOT-TIE (Basler et al., 2018). We designed gene deletion mutants using known tolerance mechanisms from the literature and complemented them with additional RNAseq analysis in response to exogenous isoprenol. We identified differential gene expression targets that were in good agreement with previous RB-TnSeq fitness profiling data (Thompson et al., 2020) (FIG. 11, panel A). From this downselection, we inactivated the pyrroloquinoline quinone (PQQ) cofactor regeneration enzyme PP_2675, as previously reported (Thompson et al., 2020), which limits most alcohol dehydrogenase activity and blocks growth on isoprenol. This approach contrasts with knocking out the approximately 20 promiscuous alcohol dehydrogenases according to UniProt and KEGG annotations. We also deleted genes encoding PP_4064-PP_4067 ivd-mccB-liuC-mccA (isovaleryl-CoA dehydrogenase, methylcrotonyl-CoA carboxylase β, methylcrotonyl-CoA hydratase, methylcrotonyl-CoA carboxylase subunit a, respectively) as these encoded proteins may enable the stepwise degradation of isoprenol into acetyl-CoA (FIG. 1, panel B) (Thompson et al., 2020). Furthermore, the ttgB homolog (PP_1385) from DOT-T1E was identified using a simple BLASTn query and deleted in IPL400. Finally, we included PP_3839 as a gene deletion target since it has known activity on other short-chain alcohols (Fong et al., 2006), even though it alone has no fitness defect on isoprenol (FIG. 11, panel B). This led us to construct two stacked deletion strains (IPL300, IPL400) with varying degrees of inactivated isoprenol-related activities compared to the WT (Materials and Methods, FIG. 1, panels B-E). IPL300 contains deletions in ΔPP_2675, ΔPP_4064-4067, and ΔPP_3839 while IPL400 also contains the same ΔPP_2675, ΔPP_4064-4067, and ΔPP_3839 deletions as well as an additional deletion in ΔttgB.

The WT and stacked deletion strains IPL300 and IPL400 were phenotypically characterized to understand their tolerance towards exogenously supplied isoprenol in batch culture (FIG. 11, panels C-F). We observed an approximately 2-fold reduction in biomass formation of the WT during the first 16 hours in M9 glucose when it was supplemented with 4 g/L isoprenol (FIG. 11, panel C). This effect became more pronounced with longer lag times at higher isoprenol concentrations (6-10 g/L). Growth completely ceased at 15 g/L. The stacked deletion strains IPL300 and IPL400 showed similar tolerance to isoprenol as the WT. IPL300 exhibited similar growth rate kinetics as the WT in the presence of 4 g/L exogenous isoprenol in glucose minimal medium (FIG. 11, panel D). However, the IPL400 strain, which has an additional ttgB deletion, displayed a shortened lag phase and higher final OD₆₀₀ values compared to both IPL300 and WT. This observation was contrary to our expectations and published reports that the strain would be more sensitive to isoprenol due to the lack of this active efflux pump (Basler et al., 2018). Both IPL300 and IPL400 were unable to grow with isoprenol as the sole carbon source in M9 media as they contained deletions of ΔPP_2675 and ΔPP_4064-ΔPP_4067, which were both shown to abolish growth individually on isoprenol when deleted (FIG. 11, panel E). These genes likely encode non-redundant activities given the observation that the ΔPP_2675 strain, but not the ΔPP_4064-ΔPP_4067 strain, essentially fully blocked isoprenol degradation (FIG. 11, panel F).

Generation of Evolved Strains with Improved Isoprenol Tolerance

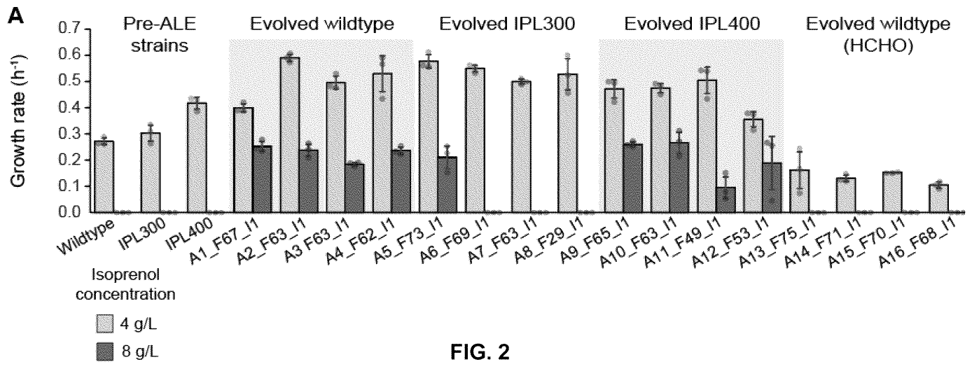

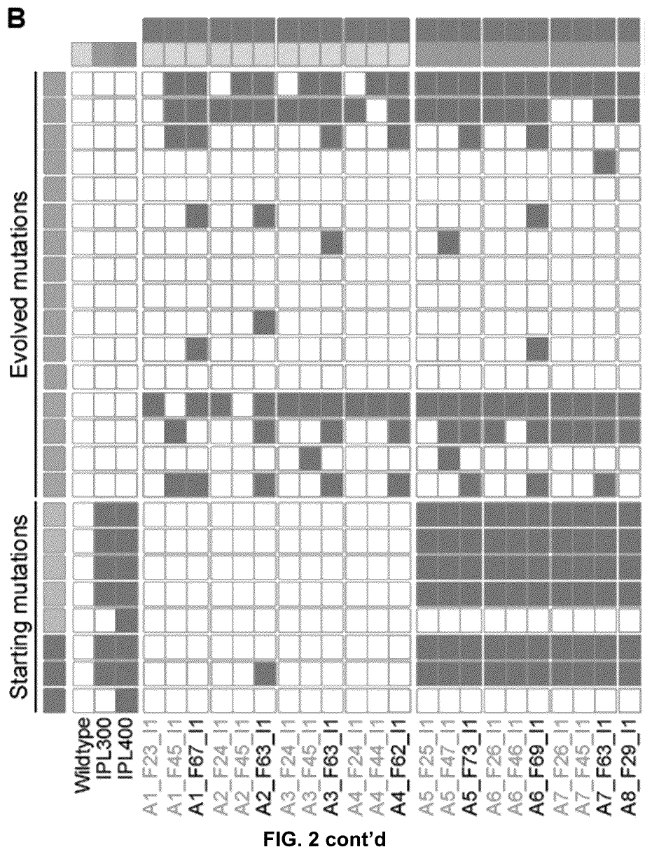

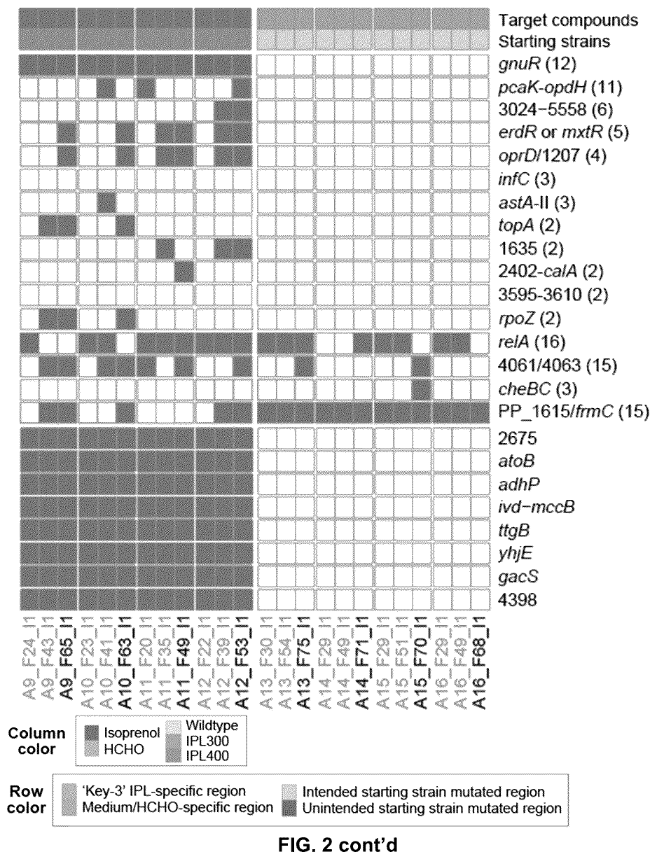

We performed high throughput TALE experiments using the WT, IPL300, and IPL400 starting strains in glucose M9 minimal medium by gradually increasing the isoprenol concentration from 4 g/L to 8 g/L in steps of 0.5-1 g/L. Each TALE experiment was conducted with four independent biological replicates in one high-throughput evolution campaign (FIG. 1, panel C and Table 4). The TALE experiments were terminated when the concentration reached 8.0 or 8.5 g/L of isoprenol, as the growth rates were reduced to approximately 0.1 h⁻¹ and no further improvements were observed in the tolerized lineages (i.e., the independent biological replicates) after approximately ~20 generations at the final isoprenol concentrations. We also tolerized the WT strain against formaldehyde stress as a control (HCHO, isolates A13-A16, FIG. 2, panels A and B) since formaldehyde is a trace impurity in commercially synthesized isoprenol used in this study (Babel and Krömer, 2020; Dyga et al., 2021). Over an approximate 2-month period, these P. putida strains underwent approximately 143 to 353 generations, which is equivalent to 1.33 to 3.32× 101 cumulative cell divisions (CCD). Similarly, for the HCHO TALE, the concentration of HCHO was increased from 0.03 g/L to 0.27 g/L over 346-387 generations, corresponding to 3.11×10¹² to 3.56×10¹² CCDs. At the end of the TALE campaign, end-point clones were isolated from the final flasks of each of the 16 lineages for further phenotype and genotype analysis.

To confirm improved fitness of the evolved isolates against their respective tolerized target compound, we compared the growth of the end-point isolates at 4 g/L or 8 g/L exogenous isoprenol with the three starting strains (isolates A1-A12, FIG. 2, panel A). Notably, all the evolved isolates displayed higher growth rates (0.50 h⁻¹, 1.8-fold increase on average) than that of the wild-type strain at 4 g/L isoprenol, the starting concentration. At 8 g/L, all evolved isolates except for three evolved IPL300 isolates (isolates A6-A8, FIG. 2, panel A) showed growth rates of up to 0.25 h⁻¹. Not only were the growth rates improved, but the average lag time was also reduced to essentially zero from 1 h in the presence of 4 g/L isoprenol. There was no strict correlation of the enhancement in the growth rates of the endpoint isolates either at 4 g/L or 8 g/L exogenous isoprenol with the strain backgrounds (isoprenol catabolismpresent or deficient). Many of the evolved IPL300 end-point isolates did not show growth at 8 g/L (FIG. 2, panel A) even though they were isolated from the populations which were exposed to essentially the same endpoint isoprenol concentration of 8.5 g/L (refer to FIG. 1, panels C-E). This phenotypic difference was likely owed to differences in the tolerance assay setup, where in the TALE regime populations were grown with constitutive and increasing concentrations of isoprenol (i.e., from an active growing state). In contrast, in the validation assay, naive cells were directly shifted to the higher 8 g/L isoprenol concentration without exposure to a lower intermediate concentration. The screened isolates tolerized against HCHO did not show improvements in tolerance to isoprenol as no HCHO-tolerant clones were cross tolerant to 8 g/L isoprenol. Overall, the validation of TALE end-point isolates using growth rate and lag time as phenotypic metrics confirmed that the isolated mutants showed up to 1.8-fold improvements for growth rate with greatly decreased lag time in the presence of isoprenol for the WT as well as the isoprenol-catabolism deficient IPL300 and IPL400 starting strains.

### Mutation Profiling of the Evolved Isolates to Reveal Mechanisms for Improved Tolerance

A mutational analysis of the evolved TALE strains was performed to link their altered phenotypes to mutated genes. After the isoprenol TALE campaign, P. putida mutants showed increased growth rates in the presence of 4 g/L isoprenol and the majority of them were capable of growing in the presence of 8 g/L, which was growth inhibitory to the starting WT, IPL300 and IPL400 strains of P. putida. The phenotypic similarity for isoprenol tolerance across multiple lineages suggested a possibility of correlating common mutations across these 12 biologically-independent lineages to identify common determinants of isoprenol tolerance. However, it was also possible that strain-specific mutants existed that enabled growth at the 8 g/L isoprenol condition in some strains, owing to the polytropic impact alcohols may have on cell membrane structure and protein denaturation. We therefore performed whole-genome sequencing of evolved isolates for a total of 46 strains (including the HCHO tolerized isolates), representing the endpoint isolates and several randomly chosen isolates from two intermediate time points per lineage (except for A8 due to a small passage number, FIG. 2, panel B). We identified a total of 158 unique mutations in 73 genetic regions (i.e., genes or intergenic regions between two genes. The evolved end-point isolates acquired 10 mutations on average. None of the strains accumulated mutations in genes responsible for mutation repair systems such as (e.g., mutL or mutS) that show such hypermutator populations a growth advantage over non-mutators, a common feature of long term ALE experiments in E. coli (Kang et al., 2019) and something that can occur at shorter ALE experiments (LaCroix et al., 2015). We also noted that the starting strains IPL300 and IPL400 also contained pre-existing mutations missing from the reference sequence: PP_4986, gacS, yhjE in IPL300, and then IPL400 had the same mutations along with a mutation in PP_4398. These mutations were likely generated during the strain construction process and point to the fact that many mutations can arise during such strain engineering campaigns.

An analysis of convergent evolution was performed to understand mutational mechanisms enabling the enhanced tolerance phenotypes. A total of 20 regions were mutated in isolates from at least two independent lineages across starting strains and for both isoprenol and HCHO. We filtered out mutations from five regions, galP-I/PP_1174 (′/′ indicates an intergenic region), PP_1325, relA, PP_3820/galU, PP_4061/PP_4063 due to their frequent occurrence in previous evolution studies with glucose-minimal media (Lim et al., 2020; Mohamed et al., 2020; Phaneuf et al., 2019) and are therefore not specifically mutated in the presence of isoprenol. Additional three regions, PP_1615/frmAC, cheBC, PP_4671, were mutated in isolates from the HCHO-tolerization experiments (FIG. 2, panel B), indicating that these regions are likely associated with tolerance mechanisms against the trace impurity HCHO. At the end of this down-selection, we identified 12 loci (gnuR, pcaK-opdH, PP_3024-PP_5558, oprD/PP_1207, erdR, mxtR, infC, astA-II, topA, PP_2402-PP_2426, PP_3595-PP_3610, rpoZ) that were highly correlated as common mutations that accumulated in response to isoprenol in the TALE experiments (FIG. 2, panel B and Table 5). Out of these 12 commonly mutated regions in response to isoprenol, we briefly discuss those mutations that occurred most frequently: gnuR, pcaK-opdH, PP_3024-PP_5558, and mxtR (in more than 4 of the 12 evolved isolates, see Table 5) as major determinants of isoprenol tolerance.

### Mutations in the Transcriptional Repressor GnuR Suggests its Relevance to Global Stress Responses to Growth Inhibitors

gnuR encodes a LacI-family transcriptional regulator (del Castillo et al., 2008) and was commonly mutated. Unique mutations were identified in all isolates from populations collected at all three-time points in the isolates A5-A12, with 100% occurrence from IPL300 and IPL400 backgrounds whereas mutations were identified only in the second and end time points of the isolates from the wild type starting strain A1-A4 (FIG. 2, panel B, Table 5). A total of 17 different mutations, including single amino acid changes, small in-/out-of-frame deletions, and early stop codon insertions, were found in its coding sequence. This observation suggested that inactivation of GnuR was helpful to achieve high fitness under the isoprenol-induced stress condition. Mutations in gnuR were similarly observed in a previous ALE study which tolerized P. putida KT2440 against triethylamine hydrogen sulfate, a protic ionic liquid (Lim et al., 2020) having a hydrocarbon chain, but at a less frequency. Its transcript levels were also significantly downregulated (log, fold<−2) when P. putida KT2440 was grown in the presence of isopentanol (Lim et al., 2022) implying that the effect of gnuR deletion is possibly involved in stress response to various growth inhibitors. Its mutation did not appear to be beneficial to HCHO stress, given no mutations were observed in isolates tolerized against HCHO.

Large Deletions in the Evolved Isolates Derived from WT and IPL300 Strains Support Fitness Advantage from Inactivating the Efflux Pump TtgABC

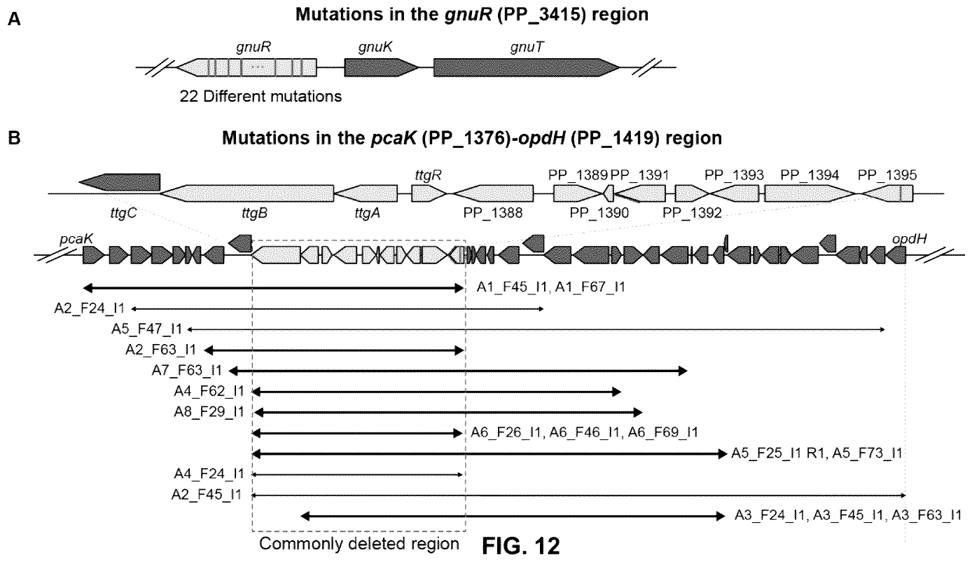

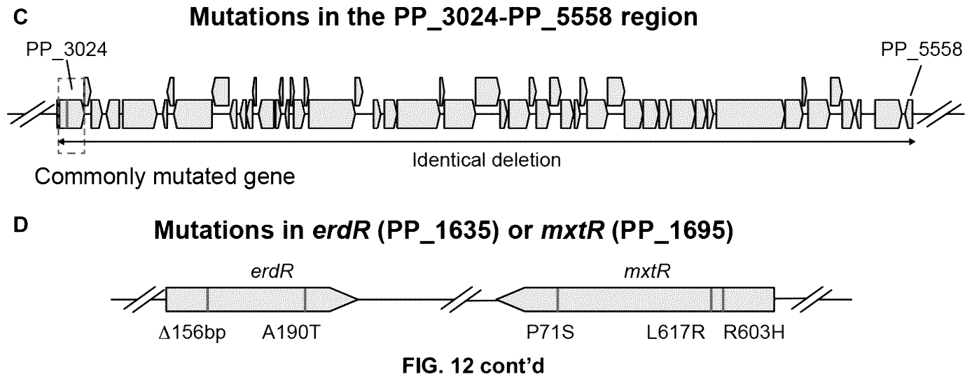

We identified mutations in nearly all the endpoint isolates (11 out of 12 isolates, 92%) either as large deletions ranging from 15-28 kb in the gene locus encoding pcaK (PP_1376) to opdH (PP_1419) in the evolved lineages from A1-A8 or a small 4 bp deletion and an SNP in PP_1395 or a SNP in PP_1396 (FIG. 2, panel B; FIG. 12, panel B) in the evolved lineages from A10-A12. The pcaK (PP_1376)-opdH (PP_1419) locus contains 45 genes encoding enzymes for the catabolism of aromatic acids (pcaK, pcaF-I, pcaTBD, pcaC, pcaP, galP-II) (Romero-Steiner et al., 1994), efflux systems and transporters (ttgABC, PP_1388, kgtP, tctABC, opdH), a putative oxaloacetate decarboxylase (PP_1389), a putative acetolactate synthase large subunit (PP_1394), regulators (ttgR, PP_1391, PP_1393, PP_1395, dctD-III), and several uncharacterized proteins (PP_1390, PP_1391, PP_1392, PP_1393). Given that most of the mutations are gene deletion resulting in loss-of-function (LoF), this implied that inactivating this large genomic locus or specific genes in this locus was beneficial for improving isoprenol tolerance. Notably, mutations in this region were different depending on whether ttgB was deleted in starting strain (FIG. 12, panel B). In the evolved isolates derived from the wildtype or IPL300 which harbors an intact ttgB, a 10-kb region of ttgA-PP_1395 was commonly deleted whereas evolved IPL400 isolates (where ttgB had been previously deleted) acquired mutations in only PP_1395 or its neighboring gene (PP_1396). This mutational pattern of losing the function of ttgB agrees with our initial observations where IPL400 showed enhanced growth in the presence of 4 g/L isoprenol (FIG. 11, panel D) over IPL300. The effect of PP_1395 is not currently clear, given the lower growth rate improvement by its deletion (see the next section). Nevertheless, taken together, these observations strongly imply that deletion of ttgABC correlates with improved fitness in the presence of isoprenol.

Large Gene Deletions in the PP_3024-PP_5558 Region Suggest the Expression of these Genes May be Disadvantageous for Cellular Fitness in the Presence of Isoprenol

It was observed that the PP_3024-PP_5558 region was mutated in 6 of the 12 (50%) end-point evolved isolates (FIG. 2, panel B). This region includes 53 phage or pyocin-related genes including phage recombinase, LysE-type translocators, an efflux transporter, regulators, a DNA-methylase, proteins coding for contractile tail particles and other hypothetical proteins with unknown functions (Weinel et al., 2002). Five of these strains (lineages A1, A3-A6) identically acquired a large deletion of 40,784 bp whereas the isolate from lineage A12 had a frame-shifting 1-bp deletion only in PP_3024 encoding a hypothetical protein. The commonality in the deletions in the PP_3024-PP_5558 region indicate that expressing these genes could be detrimental to cellular fitness when exposed to isoprenol.

### Mutations in a Two-Component System Indicate the Suppression of Unnecessary Activation of Acetate Metabolism Genes

MxtR (PP_1695 encoding a sensor kinase MxtR, also called CrbS) was known to consist of a two-component system together with ErdR (PP_1635 encoding a response regulator, also called CrbR) (Henriquez and Jung 2021). Their coding genes were mutated in five ALE experiments (ALE7 and ALE9-12) and the mutations were mostly observed in endpoint isolates. Only single amino acid change mutations occurred in MxtR (P71S, R603H, L617R), however, a 156-bp deletion or amino acid change mutation occurred in ErdR in ALE11 and ALE12. Previously, this two-component system was reported to activate genes involved in acetate utilization (Henriquez and Jung, 2021); its direct interaction with the promoter regions of yjcH-actP-I (PP_1742-3) encoding inner membrane protein and acetate permease, PP_0354 encoding CBS domain-containing protein, scpC (PP_0154) encoding propionyl-CoA: succinate CoA transferase were previously observed. Collectively, these observations implicate a potential isoprenol mechanism, suppression of adverse activation of acetate metabolism related genes in the presence of a high concentration of isoprenol.

Overall, DNA resequencing confirmed that 12 gene loci were commonly mutated in response to isoprenol. Several of the evolved isolates had accumulated mutations ranging from SNPs to large deletions in the presence of isoprenol in both the WT and the isoprenol-catabolism deficient backgrounds. These were predominantly observed in regions that encode transcriptional regulators, an efflux pump and non-essential viral proteins (gnuR, pcaK-opdH, PP_1395, PP_3024-PP_5558, mxtR and erdR). We propose that these deletions could be major determinants of isoprenol tolerance in P. putida.

### Validation of a Genetic Linkage to Isoprenol Tolerance and the Impact on Isoprenol Production by Reverse Engineering Common Mutation Targets

We validated the phenotypic linkage of isoprenol tolerance to potentially causal mutations by reverse engineering gene deletions into the WT strain. The four previously discussed regions were chosen for deletion (gnuR, ttgB-PP_1395, PP_3024-PP_5558, PP_1695) because they frequently accumulated inactivating mutations or large deletions in more than 4 ALE regimes (Table 5). We also included three specific individual genes (ttgB, PP_1395, and PP_3024) for deletion. A total of seven deletion strains were generated. The only significant increase in isoprenol tolerance for this panel of deletion mutants was in the ΔPP_3024 mutant and the larger deletion mutant containing a ΔPP_3024 deletion (i.e., the ΔPP_3024-ΔPP_5558 strain). These two mutants displayed improved growth rates by 1.7-fold on average when grown in the presence of 6 g/L isoprenol M9 media, confirming that the identified mutated regions contributed to the improved tolerance against isoprenol. As the smaller ΔPP_3024 deletion essentially accounts for most of the tolerance improvement (1.6-fold) seen in the larger region mutant. This observation implies that PP_3024 is a major player among the 53 deleted genes in the larger genetic locus. The remaining deletion mutants did show a subtle increase in growth rate but did not meet the threshold for statistical significance with p values less than 0.05. Additionally, none of these mutants showed growth on 8 g/L isoprenol (data not shown). Thus, no single mutational event emerged that could enable growth at 8 g/L as seen in the ALE strains (FIG. 2, panel A).

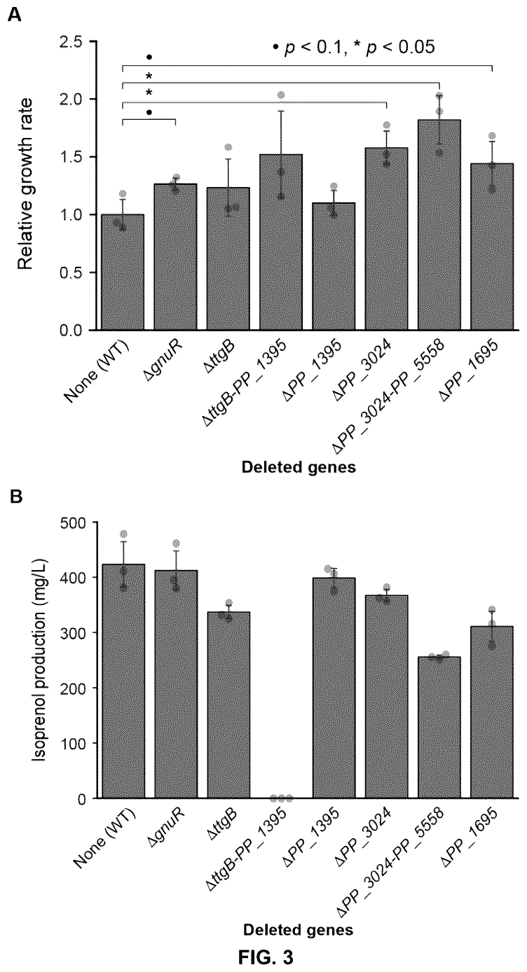

Further, we investigated the impact of these single gene deletions on isoprenol production by transforming them with the isoprenol biosynthetic plasmid, pIY670 (Banerjee et al., 2024) (FIG. 3, panel B). This plasmid contains five genes encoding a heterologous isoprenol IPP-bypass pathway. We did not observe any correlation between tolerance enhancement and isoprenol titers. Interestingly, both the large deletions mutants (PP_3024-PP_5558 and ttgB-PP_1395) showed a decrease in isoprenol titers compared to the starting strain, with the latter deletion completely abolishing isoprenol production. This finding identified a new set of genes in these regions that are directly (or) indirectly required for isoprenol production. It is also not surprising that the isoprenol titer did not increase above the control strain as the targeted mutations are not implicated in metabolic flux towards isoprenol production and/or transport, but are annotated to have ancillary regulatory or unknown functions. Reviewing both the tolerance phenotype and the isoprenol production characterization, the PP_3024 deletion mutant showed both a significant enhancement in tolerance while retaining heterologous isoprenol production compared to the starting strain and is a candidate for further study. Currently, no information is available for PP_3024, which is annotated as a putative gene with no KEGG orthology or PFam domains (Karp et al., 2019). Although some single mutation events upon reverse engineering could capture the improved tolerance profiles from lab evolution to a certain extent (Lennen et al., 2023; Lim et al., 2020; Mohamed et al., 2020), our data is consistent with isoprenol tolerance behaving as a polygenic trait that relies on the contribution of many different cellular players.3.5 Global Proteomic Profiling of Evolved Strains Identified Additional Changes Resulting from TALE

We analyzed the global effect of TALE on protein expression in the evolved strains using shotgun proteomics which revealed additional modes complementing the key genetic determinants for isoprenol tolerance. For this analysis, we chose two representative evolved end-point isolates (i.e., A10_F63_I1 and A12_F53_I1) which were derived from the same starting strain (IPL400) but contained mutations in different genes (Table 6). The two strains showed differential protein expression in about 5% of the total ~2,400 detected proteins (120 in A10_F63_I1 and 110 proteins in A12_F53_I1) in glucose minimal medium as compared to IPL400.

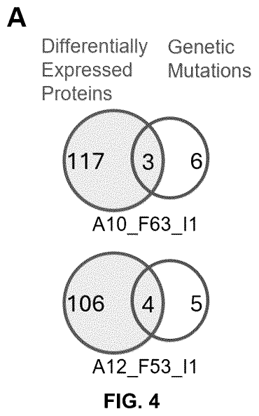

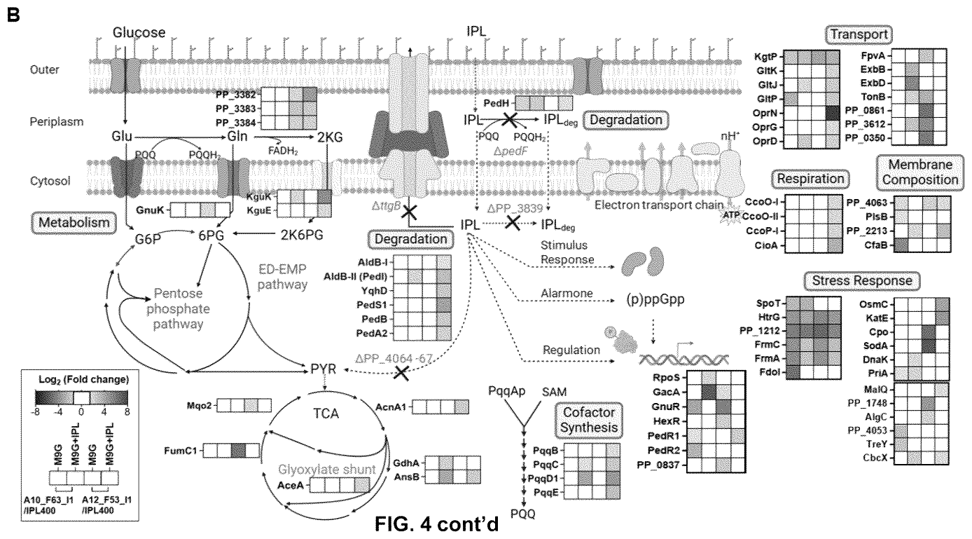

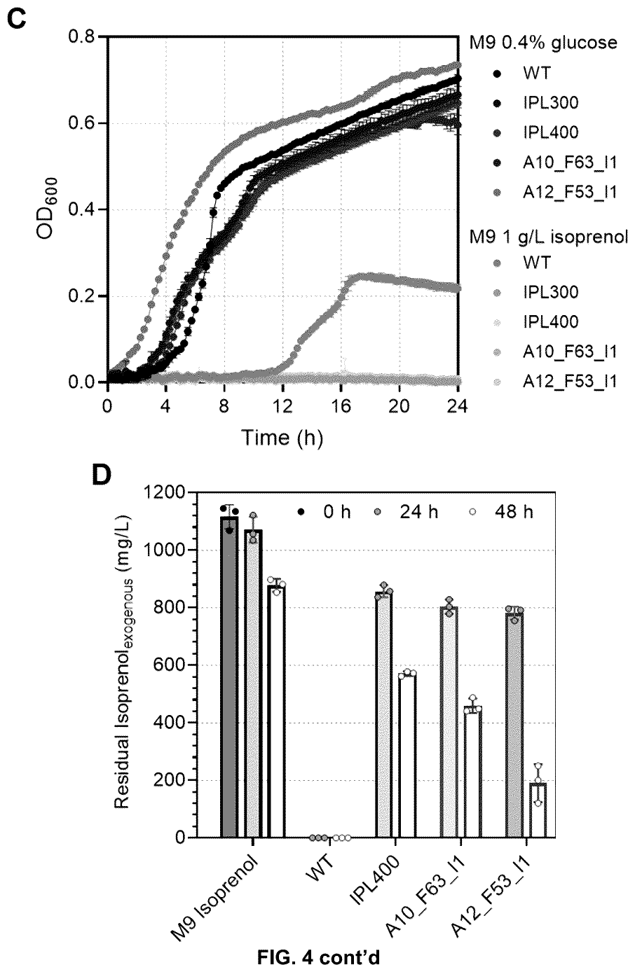

We then examined the correlation between the observed mutations from TALE and the corresponding protein levels in these two evolved strains. In A10_F63_I1, three (gnuR, PP_4063 and frmA) out of the total nine mutations, and in A12_F53_I1, four (gnuR, PP_4063, PP_3024 and frmA) out of the total nine mutations directly translated to concomitant changes in protein levels compared to the starting strain IPL400 (FIG. 4, panel A; Table 6). The remaining mutations were either not detected in the analysis or had no differential protein expression. We observed significant reduction in the protein levels due to frame-shift mutations in PP_3024 compared to the starting strain, reiterating its loss-of-function for enhanced isoprenol tolerance (Supplementary Note 1 and Table 6) and the consistency between the two-omics analyses. The levels of the transcriptional repressor GnuR (PP_3415) was also reduced in both the evolved strains as a result of SNPs, confirming its loss-of-function. An intergenic mutation in the region between PP_4061 and PP_4063 also resulted in upregulated levels of the latter coding for a putative long-chain fatty-acid-CoA ligase. This protein has also been implicated in isoprenol catabolism from Rb-TnSeq analysis albeit with a moderate fitness score (Thompson et al. 2020). The other commonly upregulated protein, FrmA (PP_1616) is not directly related to isoprenol tolerance but to the trace levels of formaldehyde in the exogenously added isoprenol during the TALE experiments and is not discussed further. Detailed analysis of the strain-specific proteomics response is described Supplementary Note 1.

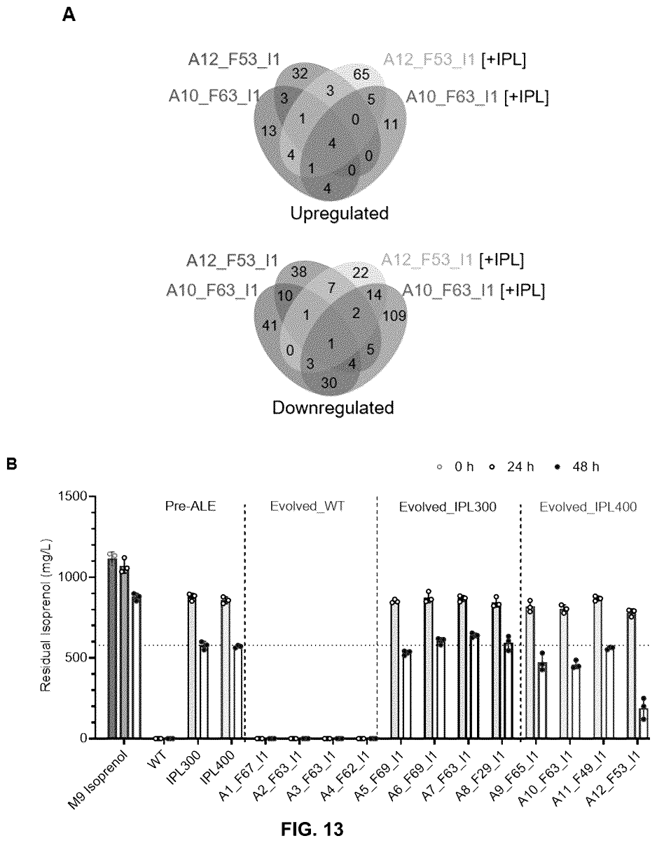

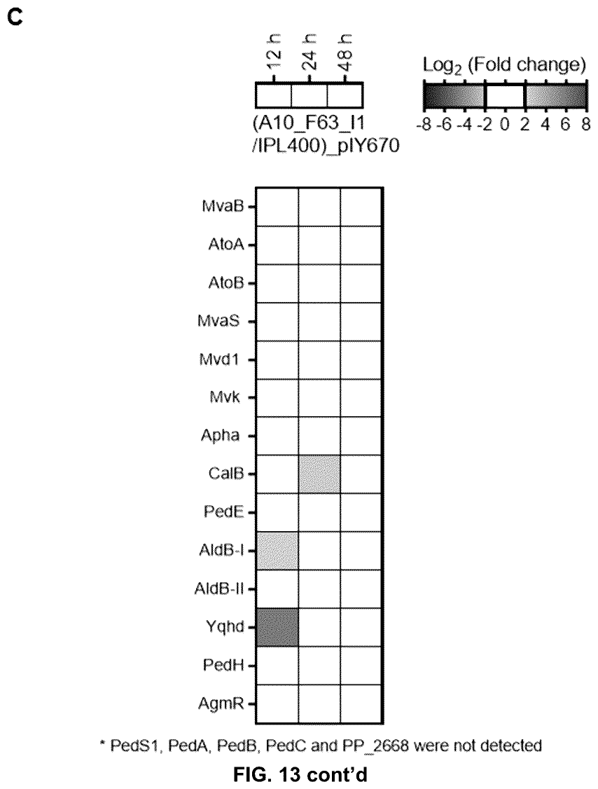

Interestingly, we also observed differential changes in protein levels coded by genes without detected mutations (FIG. 4, panel A) similar to the transcriptome response in E. coli MG1655 not directly linked to mutations in response to isoprenol stress (Babel and Krömer 2020). However, in this study, we observed only a few overlapping protein level changes between the two evolved strains and across the growth conditions (with and without isoprenol supplementation) indicating the strains' divergent responses resulting from TALE although having derived from the same parent (FIG. 13, panel A). These changes include shifts in proteins involved in metabolism, global regulation, transport, cofactor synthesis, activation of stress (or) stringent response and isoprenol degradation.

Significant downregulation of the transcriptional repressors GnuR (in both the evolved strains) and in HexR with concomitant downregulation of the ptxS regulon-PP_3382-84 and KguE (in A12_F53_I1). We also observed the significant upregulation of the glyoxylate shunt proteins (AceA and AcnA1) and the high oxygen-affinity cytochrome cbb3-1 type terminal oxidase proteins (such as CcoO-I) in the presence of isoprenol in A12_F53_I1. Both the strains also exhibited upregulation in the co-factor PQQ biosynthesis proteins (PqqB-E). Collectively, these implied enhanced glucose metabolism via the ED-EMP pathway and the cellular response to high energy demands for solvent tolerance in the evolved strains.

The evolved strain A10_F63_I1 showed upregulation of a primosome assembly protein (PriA), chaperone protein (DnaK) and exhibited a stringent response by upregulating SpoT/PP_5302 involved in (p)ppGpp biosynthesis. On the other hand, A12_F53_I1 showed significant upregulation in proteins involved in the oxidative stress response such as a hydroperoxidase (KatE), Chloroperoxidase (Cpo), Superoxide dismutase (SodA), osmotic stress response (OsmC, PP_4707), biosynthesis of osmoprotectants such as Trehalose, glycogen and NAGGN (MalQ, PP_1748, A1gC). We also observed changes in the levels of the proteins involved in composition, activation or modification of fatty acids and phospholipids (PP_4063, PlsB, PP_2213,CfaB) in addition to transporters including outer membrane porins (OprG, OprD) and those involved in iron/siderophore uptake (such as FpvA, ExbBD/TonB) in both the strains. Two of the proteins that were constitutively highly upregulated in both the strains include FmdB (PP_1212) and the signal transduction protein HtrG (PP_3631). The former protein has been observed to be important for tolerance to 1-butanol in P. putida BIRD-1 (Cuenca et al. 2016). Notably, several proteins involved in alcohol degradation (such as PedH, AldB-I, AldB-II, PedS1, PedA2) were upregulated in both the evolved isolates mostly in the presence of isoprenol. This observation suggested the possibility of alternate routes to isoprenol degradation which could have been activated by evolution and were now contributing to enhanced stress tolerance in the presence of a co-substrate regardless of their inability to utilize isoprenol as the sole carbon source (FIG. 4, panel C). Consistent with the many number of proteins upregulated, the evolved strain A12_F53_I1 showed relatively faster degradation of exogenously added isoprenol compared to the IPL400 starting strain (FIG. 4, panel D). Nevertheless, the isoprenol degradation capabilities of the other evolved strains (derived from IPL300 and IPL400) were significantly lower compared to the wildtype strain or its evolved derivatives wherein active catabolism also potentially enhances tolerance (FIG. 13, panel B).

Thus, TALE experiments with the stressor isoprenol cause mutations with both direct and indirect effects which could be elucidated by functional genomics approaches and physiological studies. These changes are consistent with well-documented solvent response of P. putida involving upregulation of DNA-repair systems, chaperones to refold denatured proteins, activation of oxidative and osmotic stress response including changes in membrane fluidity caused by adjustments in fatty acid and lipid composition and in some cases, active degradation of the target compound (Bojanovič et al. 2017; Ramos et al. 2015).

### Rational Engineering of Evolved Strains to Restore Glucose Consumption and Isoprenol Yields

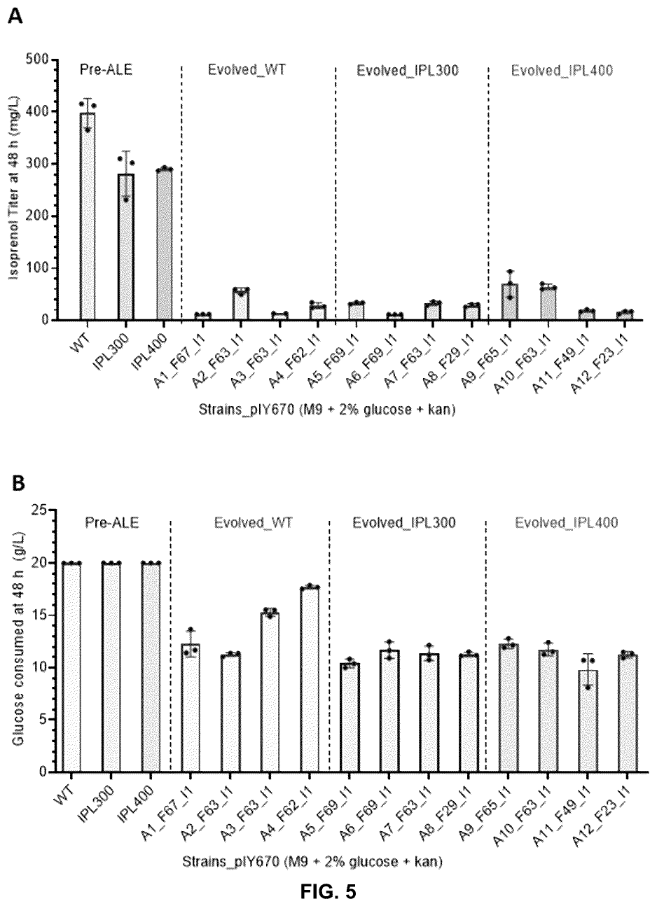

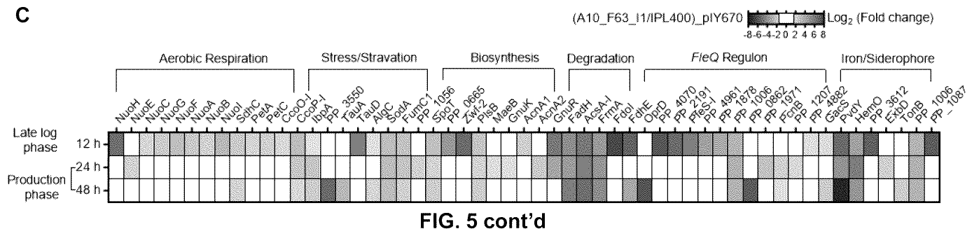

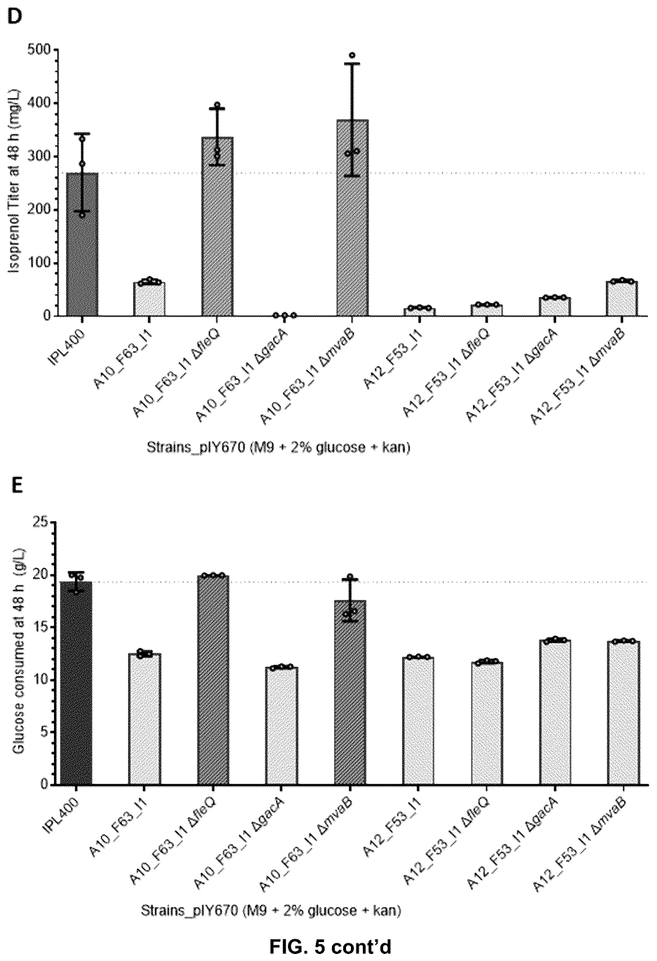

To examine the applicability of the evolved strains for use as isoprenol production chassis, we introduced the isoprenol production plasmid, pIY670 and examined their isoprenol production. Initially, all the evolved strains showed an unexpected overall reduction in isoprenol titers regardless of the starting strain background (FIG. 5, panel A) which was contrary to the generally maintained titers in the reverse engineered WT strain with the single gene deletions (FIG. 3, panel B). These decreases were likely due to significantly reduced consumption (by half) of glucose (FIG. 5, panel B). Although this suggested an overall trade-off between isoprenol production and tolerance upon TALE, the variability in degree to which isoprenol titers were reduced did not strongly correlate with the tolerance levels of these different isolates. This observation may be attributed to the polygenic nature of isoprenol tolerance, probable alterations in membrane properties, or a heightened metabolic burden following the transformation of the evolved strains with the isoprenol pathway plasmid. Among the evolved production strains, those engineered from A9_F65_I1 and A10_F63_I1 produced relatively higher concentrations of isoprenol (60-70 mg/L) at 48 hours compared to the other strains. However, this production remains significantly lower than their starting strain, which produced 280-400 mg/L at the same time point (FIG. 5, panel A).

Shotgun proteomics using a representative pre- and post-TALE strain harboring the isoprenol production plasmid helped us to further understand the limitations of heterologous isoprenol production (FIG. 5, panel C). We sampled the production strains, A10_F63_I1_pIY670 and IPL400_pIY670 at late log phase (12 h) and in the production phase (24 h and 48 h). Firstly, the low titers in this evolved production strain was not due to enhanced isoprenol degradation as we did not observe differential expression of alcohol dehydrogenases except for overexpression of CalB at 24 h. Secondly, it was not due to the poor expression of the heterologous production pathway in A10_F63_I1_pIY670 (FIG. 13, panel C). We then observed a significant reduction in the aerobic respiration pathway proteins (e.g., NuoABCEFGHI) and an increase in stress-related proteins (e.g., SpoT, SodA, IbpA) in the evolved production strain. These changes in the global proteome indicated a stringent condition, which is consistent with the impaired glucose consumption. Notably, the highest number of significantly changed proteins (Table 7, 35 genes of the 478 proteins, e.g., OpoD, PfeS-I, GacA) were found to be regulated by the global regulator fleQ, involved in flagellar and motility, adhesion, and exopolysaccharide production. The abundance of many of these proteins increased, potentially suggesting their undesired activation (FIG. 5, panel C).

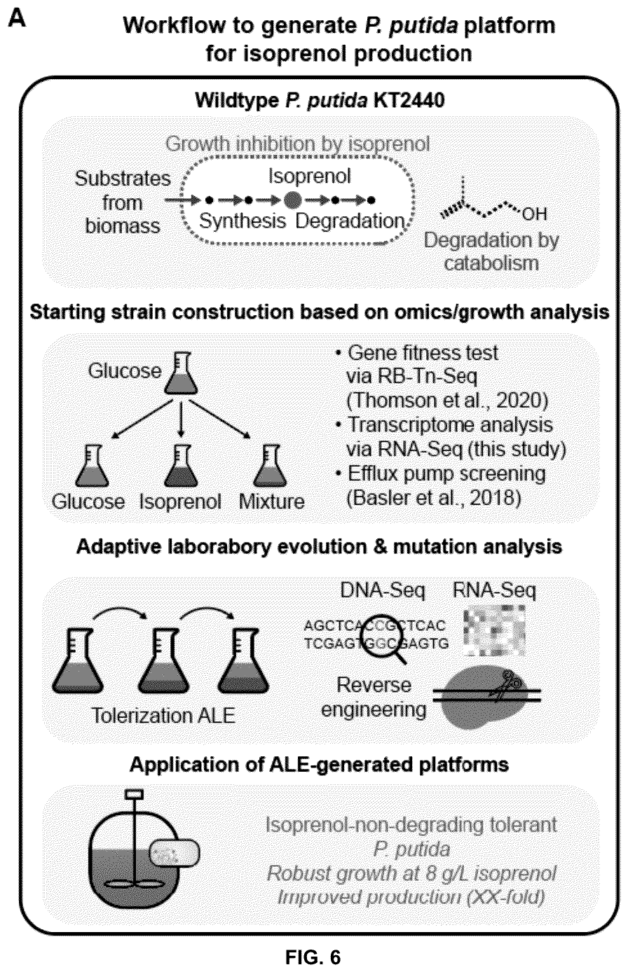

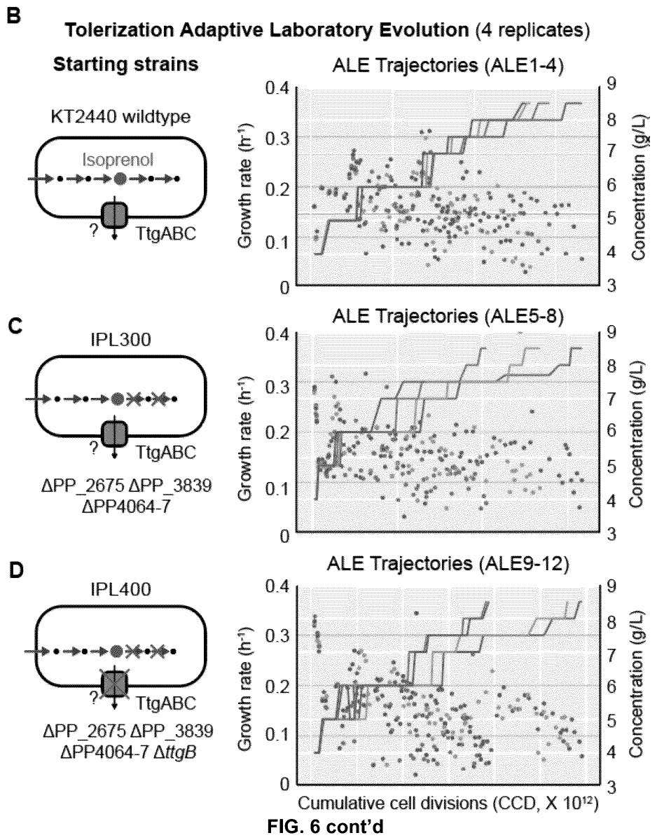

We therefore reasoned that the deletion of fleQ, would help us in leveraging the metabolic plasticity inherent to these strains overcoming any unanticipated unfavorable cellular rewiring to remedy the loss in isoprenol titers in the evolved isolate A10_F63_I1_pIY670. Previously, the deletion of fleQ has been shown to aid heterologous production by reallocation of cellular resources (Blanco-Romero et al. 2018; Kim et al. 2024), supporting our hypothesis. Indeed, the evolved isolate A10_F63_I1 ΔfleQ_pIY670 showed enhanced glucose uptake (to exhaustion at 48 h) compared to a residual glucose value of 7.5 g/L at the same time point in the starting strain thus enhancing the overall isoprenol yields (FIG. 6, panel E). The isoprenol titers also significantly increased by 4.8-fold (337 mg/L) at 48 h.

Additionally, rational engineering via a previous genome scale metabolic model guided flux analysis also helped restore isoprenol titers in the evolved engineered strain (Banerjee et al., 2024). To this end, we deleted mvaB to prevent the reverse reaction (blocking isoprenol accumulation) from 3-hydroxy 3-methylglutaryl CoA to acetoacetyl COA. Although we did not see any differential expression of MvaB in the production strain (FIG. 13), we observed that the A10_F63_I1 ΔmvaB_pIY670 strain now produced 370 mg/L of isoprenol up from 70 mg/L (FIG. 5, panel D). However, this isolate did not restore complete glucose utilization at 48 h (FIG. 5, panel E) suggesting that deletion of mvaB alone is not sufficient to rewire the impeded carbon metabolism back to pre-ALE levels.

Thus, rational strain engineering of the tolerized isolate A10_F63_I1 resulted in engineered strains that were now both tolerant to up to 8 g/L isoprenol whilst producing significant isoprenol titers as the starting strains. On the other hand, none of the deletion strains from the A12 lineage showed any dramatic improvement in isoprenol titers (FIG. 5, panel D). This further substantiated our observations from the proteomics datasets in the prior section that these two isolates evolved differentially in response to an exogenous isoprenol stress.

### CONCLUSIONS

Enhancing microbial tolerance is a necessary approach when developing efficient biofuel production processes. When isoprenol is converted to the bio-jetfuel DMCO, our recent technoeconomic analysis for commercial viability of this process demands that isoprenol be generated from lignocellulosic sugars at 90% theoretical yield or higher (Baral et al. 2021; Baral et al. 2023). Using current growth conditions and yield calculations (Banerjee et al., 2024), this would be equivalent to producing equal or greater than 6.2 g/L isoprenol from 20 g/L glucose. In this study, we successfully applied the TALE approach to enhance the tolerance of P. putida KT2440 and its genetically engineered derivatives to isoprenol, achieving stable growth at concentrations as high as 8 g/L. At first glance, this would satisfy the criteria to be useful for microbial isoprenol bioconversion and even surpasses the 6.8 g/L limit reported in a study in another gram-negative host, E. coli K-12 MG1655 (Babel and Krömer, 2020). Integrating process strategies such as two-phase extraction systems or in-situ alcohol removal could further improve viability and scalability to meet productivity targets from complex carbon streams. Thus, the TALE approach was successful in meeting a tolerance benchmark related to economic feasibility.

This work provides a comprehensive experimental mutational landscape to identify key genetic determinants of isoprenol tolerance by physiological and genomic analyses from three starting genotypes. One finding was increased isoprenol degradation in a subset of evolved lineages. However, growth analysis on isoprenol and/or with a co-substrate glucose in the starting strains and some of the evolved isolates showed that the catabolism of isoprenol as a carbon source in P. putida was not always particularly relevant to the resistance phenotype, unlike ethanol in E. coli (Goodarzi et al., 2010). In addition, only one of the evolved isolates (A12_F53_I1) showed significant enhancement (67%) in degrading isoprenol to a yet-unknown product over its catabolism-mitigated starting strain IPL400. Other characterized isolates had less of an increase in degradation. While no mutations in catabolic enzymes were detected from whole genome resequencing to support this claim, comparative proteomics analysis pinpointed several upregulated enzymes (such as AldB-I, AldB-II and PP_2668) that may have promiscuous alcohol degradative activity towards isoprenol in this evolved strain.

Mutations in gnuR, ttgB-PP_1395 and PP_3024-PP_5558 in the evolved strains resulted in clear loss-of-function mutations and these findings were supported by a concomitant decrease in protein levels. Some intergenic mutations such as in PP_4063 could have resulted in enhancing mutations (supported by increased protein abundance) while the effect of others (such as SNP in PP_1695) could not be verified based on our current analysis and warrants further characterization based on activity assays. One of the most notable outcomes of our study is the improved isoprenol tolerance via inactivation of TtgB in strains where it was originally functional. Contrary to many efflux pump studies such as that of AcrB in E. coli (Fisher et al. 2014) which demonstrated improved susceptibility (and thereby reduced tolerance) to solvents upon its deletion, our observation highlights the genetic plasticity of gram-negative microbes to alcohol tolerance, as the efflux pump TtgB has also been implicated in enhanced tolerance to isoprenol and n-butanol in P. putida (Cuenca et al. 2016; Basler et al. 2018). Although reverse engineering (via generation of isogenic deletions in the WT strain) indicated a combinatorial effect of multiple mutations to achieve the tolerance limits observed through TALE, deleting PP_3024 and ttgB in the chassis strains demonstrating gram-scale production (Banerjee et al. 2024) could be a rational next step. In addition, the isoprenol tolerance mechanisms identified here using genomics and comparative proteomics also showed little overlap with those observed in the E. coli study (Babel and Krömer, 2020). In E. coli, isoprenol tolerance was linked to mutations in membrane-associated genes (fatty acid synthesis) and membrane integrity while P. putida inactivated efflux pumps and undesired genes.

While the increased isoprenol tolerance in evolved strains generated in this study was promising, evolved strains did not show a corresponding benefit towards producing isoprenol when transformed with a heterologous pathway. However, guided by proteomics analysis and prior research, we restored isoprenol production levels by deleting the biofilm and flagella master regulator, fleQ and mvaB respectively, yielding a strain that was both tolerant to isoprenol and similarly functional as a chassis compared to the starting strain for isoprenol production. Ideally, the mutations enriched by TALE would engender non-degradative mechanisms for product tolerance. There are some exemplary ALE-driven reports where this duality is observed—for native yeast ethanol production (Mavrommati et al., 2023), L-serine production in E. coli (Mundhada et al., 2017) or for heterologous isobutyrate and 2,3-butanediol production in E. coli (Lennen et al., 2023). However, given the indication that isoprenol degradation is a possible tolerance mechanism, further iterative engineering cycles are likely warranted to understand the genetic basis of the enhanced degradation in an effort to eliminate it, as this tolerance mechanism is antagonistic to production.

In summary, the TALE approach successfully resulted in robust isoprenol tolerized strains. The enhanced tolerance can be significantly advantageous to further develop chassis with superior titers, rates, and yields, which are key factors in determining overall commercial viability. Ultimately, the development of such resilient strains can accelerate the transition to sustainable aviation fuels by making biobased isoprenol production more economically feasible and competitive with conventional petroleum-derived fuels. As such, this advancement represents a key step toward complementing the use of fossil fuels and mitigating the environmental impact of the aviation sector.

### Supplementary Note 1. Detailed Proteomics Characterization of the Evolved Strains.

Protein Levels that Directly Correlated to Mutations Acquired During TALE in the Evolved Strains A10_F63_I1 and A12_F53_I1

In both the evolved strains, some mutated genes were differentially expressed under standard laboratory growth conditions (without exogenous isoprenol addition, Table 6). Not all mutants could be characterized due to inherent limitations in the shotgun proteomics detection method. Several proteins of note are described here. The basic amino acid porin encoded by oprD (PP_1206) with a C→T SNP in the intergenic oprD/PP_1207 (-360/-331) region showed a significant increase in protein levels in the presence of isoprenol in both the evolved strains. As briefly mentioned in the resequencing section earlier, the intergenic region between frmA and PP_1615 also contained a SNP; correspondingly, we observed a constitutive increase of FrmA protein expression in both strains. The transcriptional repressor GnuR (PP_3415) showed significantly lower protein levels on glucose minimal medium. This downregulation could be related to changes in protein activity from two substitution mutants (V46I and G167R). The V46I mutation (in isolate A12_F53_I1) lies in a probable DNA binding domain and G167R (in isolate A10_F63_I1) lies in a substrate recognition domain (Finn et al., 2011). We also observed significant constitutive downregulation in the AraC family transcriptional regulator PP_1395 in isolate A12_F53_I1, where genome resequencing indicated point mutation corresponding to an L81R amino acid mutation in a putative ligand binding domain could change protein stability or abundance. Finally, in the same isolate, a 1-bp deletion in the coding region at basepair 167 in the PP_3024 coding sequence would result in an early ribosome termination where the frameshift mutation would generate a premature stop codon. These analyses corroborated the downstream impact on protein levels as hinted by the DNA resequencing, but the change in protein abundance could not be predicted by the sequence information alone.

### Additional Proteomic Responses in the Evolved Strains in Response to Isoprenol Stress

Upon isoprenol supplementation, ~8% of the detected proteins were differentially expressed in the both the evolved strains compared to IPL400 (FIG. 13, panel A). This clearly indicated proteomic shifts in response to isoprenol stress which could be either direct or indirect consequences of mutations resulting from TALE. The majority of differentially abundant proteins were unique to each isolate. Specifically, the isolate A10_F63_I1 has a significant upregulation of the stringent response regulator SpoT/PP_5302 (on both minimal medium and upon isoprenol supplementation). In this isolate, the master regulator of stringent response involved in (p)ppGpp synthesis, RelA was not affected in its gene sequence (FIG. 2, panel B) or in its protein levels but the upregulation of its complement SpoT (which is a (p)ppGpp hydrolase with a weak synthase activity) was observed. This could induce large-scale transcriptional changes leading to activation of various stress response regulons. In addition, (p)ppGpp directly inhibits other enzymes thereby reallocating cellular resources towards adaptation and survival under stressful conditions (Gaca et al., 2015). On the other hand, the two-component system regulator GacA and its regulon, the sigma factor RpoS, were both significantly downregulated only in the presence of isoprenol in this isolate.

While the isolate A12_F53_I1 showed more downregulated genes than A10_F63_I1, they could not be easily categorized into subsystems. We thus focused on the upregulated genes in this isolate. Many alcohol metabolism related proteins such as dehydrogenases, two component signaling systems, associated transporters and associated cofactor synthesis proteins were differentially upregulated in the presence of isoprenol. Apart from PedH, AldB-II and OprD that were also observed in A10_F64_I1, we also observed upregulation of the aldehyde dehydrogenase AldB-I (FIG. 4, panel B). It was also interesting to note the significant upregulation of the isoprenol responsive two-component system sensor histidine kinase/response regulator PedS1/PP_2664 in this isolate. In addition, PedB and PedA2 are ABC transporters reported to be involved in efflux of chloramphenicol, aromatics and more recently lanthanides (Fernández et al., 2012; Wehrmann et al., 2019) and were upregulated in this isolate. Transposon mutants in coding sequences for these proteins also have negative fitness when grown on isoprenol (Thompson et al. 2010) suggesting that they could facilitate efflux or transport of isoprenol (complementing for the loss of ttgB) or contribute to stabilize membrane architecture with other upregulated outer membrane proteins (OprD, OprG). The most notable difference between the two evolved strains is the upregulation in aerobic respiration proteins-CcoO-I, II and CcoP-I (FIG. 4, panel B) in A12_F53_I1 which has been previously observed in P. putida in response to solvent stress such as 1-butanol and octane.

TABLE 2
Strains and plasmids used in this study.

| Name | Genotype with notes | Reference |
|---|---|---|
| Strains | | |
| KT2440 | Wild-type P. putida KT2440 | ATCC 47054 |
| E. coli S-17 | E. coli biparental conjugation strain | ATCC 47055 |
| KT2440 ΔPP_2675 | KT2440 with the complete internal in-frame deletion | (Thompson et |
| A14-PP_2676 (JBEI- | of PP_2675 and partial truncation of the first fourteen | al., 2020) |
| 147164) | amino acids of PP_2676 | |
| KT2440 ΔPP_3839 | KT2440 with the complete internal in-frame deletion | (Thompson et |
| (JBEI-147168) | of PP_3839 | al., 2020) |
| KT2440 ΔPP_4064- | KT2440 with the complete internal in-frame deletion | (Thompson et |
| 4067 | of PP_4064-4067 | al., 2020) |
| (JBEI-147167) | | |
| KT2440 ΔPP_2675 | KT2440 ΔPP_2675 A14-PP_2676 ΔPP_3839 | (Thompson et |
| A14-PP_2676 | | al., 2020) |
| ΔPP_3839 | | |
| (JBEI-147169) | | |
| IPL300 | KT2440 ΔPP_2675 A14-PP_2676 ΔPP_3839 | This study |
| (JBEI-264601) | ΔPP_4064-ΔPP_4067 | |
| IPL400 | KT2440 ΔPP_2675 A14-PP_2676 ΔPP_3839 | This study |
| (JBEI-264602) | ΔPP_4064-ΔPP_4067 ΔttgB (PP_1385) | |
| A1_F67_I1 | An evolved wildtype isolate from ALE1 | This study |
| (JBEI-264551) | | |
| A2_F63_I1 | An evolved wildtype isolate from ALE2 | This study |
| (JBEI-264552) | | |
| A3_F63_I1 | An evolved wildtype isolate from ALE3 | This study |
| (JBEI-264553) | | |
| A4_F62_I1 | An evolved wildtype isolate from ALE4 | This study |
| (JBEI-264554) | | |
| A5_F73_I1 | An evolved IPL300 isolate from ALE5 | This study |
| (JBEI-264555) | | |
| A6_F69_I1 | An evolved IPL300 isolate from ALE6 | This study |
| (JBEI-264556) | | |
| A7_F63_I1 | An evolved IPL300 isolate from ALE7 | This study |
| (JBEI-264557) | | |
| A8_F29_I1 | An evolved IPL300 isolate from ALE8 | This study |
| (JBEI-264558) | | |
| A9_F65_I1 | An evolved IPL400 isolate from ALE9 | This study |
| (JBEI-264559) | | |
| A10_F63_I1 | An evolved IPL400 isolate from ALE10 | This study |
| (JBEI-264560) | | |
| A11_F49_I1 | An evolved IPL400 isolate from ALE11 | This study |
| (JBEI-264561) | | |
| A12_F53_I1 | An evolved IPL400 isolate from ALE12 | This study |
| (JBEI-264562) | | |
| A13_F75_I1 | An evolved wildtype isolate from ALE13 | This study |
| (JBEI-234918) | | |
| A14_F71_I1 | An evolved wildtype isolate from ALE14 | This study |
| (JBEI-234919) | | |
| A15_F70_I1 | An evolved wildtype isolate from ALE15 | This study |
| (JBEI-234920) | | |
| A16_F68_I1 | An evolved wildtype isolate from ALE16 | This study |
| (JBEI-234921) | | |
| KT2440 ΔgnuR | KT2440 ΔgnuR (PP_3415) | This study |
| (JBEI-235864) | | |
| KT2440 ΔttgB | KT2440 ΔttgB (PP_1385) | This study |
| (JBEI-235865) | | |
| KT2440 ΔttgB_L | KT2440 ΔPP_1385-1395 | This study |
| (JBEI-235866) | | |
| KT2440 ΔPP_1395 | KT2440 ΔPP_1395 | This study |
| (JBEI-235867) | | |
| KT2440 ΔPP_3024 | KT2440 ΔPP_3024 | This study |
| (JBEI-235868) | | |
| KT2440 ΔPP_3024- | KT2440 ΔPP_3024-ΔPP_3067 ΔPP_5558 | This study |
| ΔPP_5558 | | |
| (JBEI-235869) | | |
| KT2440 ΔmxtR | KT2440 ΔmxtR (PP_1695) | This study |
| (JBEI-235870) | | |
| A10_F63_I1 ΔfleQ | Evolved isolate A10_F63_I1 ΔfleQ (PP_4373) | This study |
| (JBEI-264563) | | |
| A10_F63_I1 ΔmvaB | Evolved isolate A10_F63_I1 ΔmvaB (PP_3540) | This study |
| (JBEI-235870) | | |
| A10_F63_I1 ΔgacA | Evolved isolate A10_F63_I1 ΔgacA (PP_4099) | This study |
| (JBEI-264565) | | |
| A12_F53_I1 ΔgacA | Evolved isolate A12_F53_I1 ΔgacA (PP_4099) | This study |
| (JBEI-264571) | | |
| A12_F53_I1 ΔagmR | Evolved isolate A12_F53_I1 ΔagmR (PP_2665) | This study |
| (JBEI-264569) | | |
| A12_F53_I1 ΔmvaB | Evolved isolate A12_F53_I1 ΔmvaB (PP_4099) | This study |
| (JBEI-235870) | | |
| Plasmids | | |
| pMQ30 gnuR | Allelic exchange plasmid for deleting gnuR | This study |
| (JBEI-235873) | | |
| pMQ30 ttgB_L | Allelic exchange plasmid for deleting PP_1385-95 | This study |
| (JBEI-235875) | | |
| pMQ30 PP_1395 | Allelic exchange plasmid for deleting PP_1395 | This study |
| (JBEI-235876) | | |
| pMQ30 PP_3024 | Allelic exchange plasmid for deleting PP_3024 | This study |
| (JBEI-235877) | | |
| pMQ30 PP_3024-L | Allelic exchange plasmid for deleting PP_3024- | This study |
| (JBEI-235878) | PP_5558 | |
| pMQ30 PP_3839 | Allelic exchange plasmid for deleting PP_3839 | (Thompson et |
| (JBEI-109853) | | al., 2020) |
| pMQ30 PP_4064- | Allelic exchange plasmid for deleting PP_4064- | (Thompson et |
| 4067 | PP_4067 | al., 2020) |
| pTE289 | pTE289 barcoded ΔttgB PP_1385 Kan SacB | (Mohamed et |
| (JBEI-108734) | | al., 2020) |
| pIY670 | Isoprenol production plasmid | (Banerjee et |
| (JBEI-264945) | pRK2-Kan-araC-P_{BAD}-MvaS_{ef}-MvaE_{ef}-T_{rpoH}-P_{trc1-O}- | al., 2020) |
| | MKₘₘ-PMD_{HKQ}-AphA | |
| pAO1/pTE452 | Recombineering plasmid | (Wannier et |
| | PORTMAGE-Pal-recT-mutL-E36K aaaC1 (gntR) | al., 2020) |
| pTE433β | gRNA plasmid (Cpf1_{Fn} containing) backbone for | (Eng et al., |
| (JBEI-236684) | recombineering | 2023) |
| | P_{LacM}-RBSₒₚₜ-Cpf1-P_{J23119}-gRNA insertion site-LacI- | |
| | BBR1-kanR | |
| pTE490 | gRNA plasmid targeting PP_4373 (fleQ) P_{LacM}- | (Eng et al., |
| (JBEI-236853) | RBSₒₚₜ-Cpf1-P_{J23119}-PP_4373-gRNA-LacI-BBR1- | 2023) |
| | kanR | |
| pTE535 | gRNA plasmid targeting PP_2665 (agmR/pedR1) | This study |
| (JBEI-264947) | P_{LacM}-RBSₒₚₜ-Cpf1-P_{J23119}-PP_2665-gRNA-LacI- | |
| | BBR1-kanR | |
| pTE479 | gRNA plasmid targeting PP_4099 (gacA) | This study |
| (JBEI-264949) | P_{LacM}-RBSₒₚₜ-Cpf1-P_{J23119}-PP_4099-gRNA-LacI- | |
| | BBR1-kanR | |
| pTE769 | gRNA plasmid targeting PP_3540 (mvaB) | This study |
| (JBEI-264951) | P_{LacM}-RBSₒₚₜ-Cpf1-P_{J23119}-PP_3540-gRNA-LacI- | |
| | BBR1-kanR | |

TABLE 3
Oligonucleotides used in this study.

| Name | Sequence (5′-3′)ª |
|---|---|
| pTE275_BB_only_F | CGACCAATTGCTCAAAGCCT (SEQ ID NO: 1) |
| pTE275_BB_only_B | CATGTATACTAAACTCACAAATTAGAGC (SEQ ID NO: 2) |
| pTE275_ttgB_L_F | GCTCTAATTTGTGAGTTTAGTATACATGTCTGCAGGAACA |
| | GGTACATCAC (SEQ ID NO: 3) |
| pTE275_ttgB_L_B | AGAGGCTTTGAGCAATTGGTCGGAATTCCCTACGTGCCAT |
| | GTTG (SEQ ID NO: 4) |
| pTE275_PP_1395_F | GCTCTAATTTGTGAGTTTAGTATACATGATCCATACCCCG |
| | GATTTCATCG (SEQ ID NO: 5) |
| pTE275_PP_1395_B | AGGCTTTGAGCAATTGGTCGGAATTCCCTACGTGCCATGT |
| | TG (SEQ ID NO: 6) |
| pTE275_PP_3024_F | GCTCTAATTTGTGAGTTTAGTATACATGATTACAGGCTCC |
| | TCGGTATTGC (SEQ ID NO: 7) |
| pTE275_PP_3024_B | AGGCTTTGAGCAATTGGTCGACTCACACCTGATGCGTATC |
| | TC (SEQ ID NO: 8) |
| pTE275_PP_3024_L_B | AGGCTTTGAGCAATTGGTCGTATCTACGTAAGCCATAGCC |
| | GCC (SEQ ID NO: 9) |
| gRNA_PP_1695_F | GATCCGAATTTCTACTGTTGTAGATGGCGTTCCTGCCCA |
| | TCTACCCAAATAAAACGAAAGGCTCAGTCGA (SEQ ID |
| | NO: 10) |
| gRNA_PP_1695_B | TCGACTGAGCCTTTCGTTTTATTTGGGTAGATGGGCAGG |
| | AACGCCATCTACAACAGTAGAAATTCGGATC (SEQ ID |
| | NO: 11) |
| gRNA_PP_4099 | AGGTATAATGGATCCGAATTTCTACTGTTGTAGATAGTGC |
| | CGACTCGCCGGAATCTATTTCAAATAAAACGAAAGGCT |
| | CAGTCGAAAGACT (SEQ ID NO: 12) |
| gRNA_PP_4373 | GGTATAATGGATCCGAATTTCTACTGTTGTAGATCTATTG |
| | CTGTTTGGGCTGGGTCAAATAAAACGAAAGGCTCAGTC |
| | GAAAGACTGGG (SEQ ID NO: 13) |
| gRNA_PP_2665 | AGGTATAATGGATCCGAATTTCTACTGTTGTAGATGCGAA |
| | GCCATCCACAATGTCAATTTCAAATAAAACGAAAGGCTC |
| | AGTCGAAAGAC (SEQ ID NO: 14) |
| gRNA_PP_3540 | AGGTATAATGGATCCGAATTTCTACTGTTGTAGATGTGTC |
| | CCCCAAGTGGGTGCCAAAATAAAACGAAAGGCTCAGTC |
| | GAAAGACTGGGC (SEQ ID NO: 15) |
| ssDNA oligo_PP_1695 | TCGTTGTCCAGCGGGCTGATCGCCGTGGTCGCCCTGGCCT |
| | ATAACCGGTCAAGCCTGCGGCCTTGCGCGCCCTGCTCAAT |
| | CGCCATC (SEQ ID NO: 16) |
| ssDNA oligo_PP_4099 | TGTTAAGGTGGCGGGATCCAATCGCCCTGCTGACGAGGT |
| | GTGTGCAACCTCATGTCCCAAGTCTTTGATGCCAGCGCAT |
| | TCCTGGCGACC (SEQ ID NO: 17) |
| ssDNA oligo_PP_4373 | CACGCCCCTGCAGGGCGCCGATATGACTAGGGAAGTTGC |
| | TATTGCTGTTTGGGCTGGGTGTTCGCCTTGGGGGTTGCGT |
| | TTGGGCGGGGT (SEQ ID NO: 18) |
| ssDNA oligo_PP_2665 | GTATGCGTGCCCCGCTCCATCCAATAAAAACAAGGTGAA |
| | AAGGCCGCTGTTCCGGCCGCTTCGCGGATCAACCCGCGA |
| | AGCGGCCAACCG (SEQ ID NO: 19) |
| ssDNA oligo_PP_3540 | CCCTGAACAACAAAGACAACCGCTGAACGCCACAGGAAA |
| | CCCGCCGTCACACGGGTGTCACATTGGTGTGGGTGAAGTG |
| | TTACCTCCCGC (SEQ ID NO: 20) |

^{a}Bold font indicates gRNA targeting sequence. A 5′ TTTN PAM sequence was used to identify compatible Cas12a/Cpf1cut sites.

TABLE 4
Summary of the Tolerization ALE (TALE) to isoprenol.

| | | | | Initial | | | Final | | | |
|---|---|---|---|---|---|---|---|---|---|---|
| | | Starting | | growth | Ending | | growth | Passages | | CCD |
| | ALE# | concentration | | rate (h⁻¹) | concentration | | rate^{b} (h⁻¹) | # | Generations | (10¹²) |
| Isoprenol | 1 | 4 | g/L | 0.152 | 8.5 | g/L | 0.113 | 67 | 353 | 3.32 |
| TALE | 2 | | | 0.152 | 8.5 | g/L | 0.073 | 64 | 320 | 2.68 |
| KT2440 | 3 | | | 0.153 | 8.5 | g/L | 0.095 | 63 | 325 | 2.92 |
| | 4 | | | 0.157 | 8.5 | g/L | 0.057 | 63 | 313 | 2.63 |
| Isoprenol | 5 | 4 | g/L | 0.253 | 8.5 | g/L | 0.091 | 74 | 320 | 3.17 |
| TALE | 6 | | | 0.253 | 8.5 | g/L | 0.095 | 70 | 341 | 2.68 |
| IPL300 | 7 | | | 0.252 | 8.5 | g/L | 0.060 | 64 | 297 | 2.05 |
| | 8* | | | 0.252 | 7.5 | g/L | 0.137 | 29 | 143 | 1.33 |
| Isoprenol | 9 | 4 | g/L | 0.300 | 8.5 | g/L | 0.085 | 67 | 340 | 2.96 |
| TALE | 10 | | | 0.300 | 8 | g/L | 0.119 | 66 | 326 | 2.80 |
| IPL400 | 11 | | | 0.305 | 8 | g/L | 0.062 | 51 | 244 | 1.91 |
| | 12 | | | 0.298 | 8 | g/L | 0.061 | 55 | 256 | 1.94 |
| HCHO | 13 | 1 | mM | 0.284 | 9 | mM | 0.197 | 75 | 387 | 3.56 |
| TALE | 14 | | | 0.247 | 9 | mM | 0.204 | 71 | 363 | 3.35 |
| KT2440 | 15 | | | 0.294 | 9 | mM | 0.144 | 70 | 362 | 3.41 |
| | 16 | | | 0.288 | 9 | mM | 0.157 | 68 | 346 | 3.11 |

^{a}Average growth rate, observed in the three first flasks of each experiment.
^{b}Average growth rate, observed in the three last flasks of each experiment.
*Has a lower passage than other lineages since the experiment was restarted from an early intermediate stock due to cross-contamination.

TABLE 5
Commonly Mutated Isoprenol Tolerance Specific Regions.

| | | | Mutation | # of | ALE |
|---|---|---|---|---|---|
| # | Genetic region^{a} | Product | type | ALE | condition |
| 1 | gnuR | Transcriptional regulator, LacI | SNP, in- | 12 | A1-4, A5-8, |
| | (PP_3415)^{a} | family | frame | | A9-12 |
| | | | deletion, | | |
| | | | frame-shift | | |
| | | | deletion, | | |
| | | | early | | |
| | | | termination | | |
| 2 | pcaK | 22 Genes | Deletion | 11 | A1-4, A5-8, |
| | (PP_1376)- | | | | A10-12 |
| | opdH^{a} | | | | |
| 3 | PP_3024- | 53 Genes | Deletion | 6 | A1, A3-6, |
| | PP_5558 | | | | A12 |
| 4 | erdR (PP_1635) | LuxR family transcriptional | Deletion, | 5 | A7, A9-12 |
| | or mxtR | | | | |
| | (PP_1695) | regulator or putative Sodium- | SNP | | |
| | | solute symporter/sensory box | | | |
| | | histidine kinase/response | | | |
| | | regulator | | | |
| 5 | oprD | Basic-amino-acid specific porin | SNP | 4 | A9-12 |
| | (PP_1206)/ | OprD/conserved protein of | | | |
| | PP_1207 | unknown function | | | |
| 6 | infC (PP_2466) | Translation initiation factor IF- | In-frame | 3 | A1-2, A6 |
| | | 3 | deletion | | |
| 7 | astA-II | Arginine N-succinyltransferase, | Early | 3 | A3, A5, A10 |
| | (PP_4480) | subunit alpha | termination, | | |
| | | | SNP | | |
| 8 | topA (PP_2139) | DNA topoisomerase I | SNP | 2 | A9 and A10 |
| 9 | PP_2402-calA | 25 Genes^{a} | Deletion, | 2 | A2 and A11 |
| | (PP_2426) | | frameshift | | |
| | | | (PP_2425) | | |
| 10 | PP_3595- | 17 Genes^{a} | Deletion | 2 | A1 and A6 |
| | PP_3610 | | | | |
| 11 | rpoZ (PP_5301) | DNA-directed RNA | Frameshift | 2 | A9 and A10 |
| | | polymerase subunit omega | | | |

^{a}Full gene lists are given in Supplementary Data 1.

TABLE 6
Details of the mutations in the A10_F63_I1 and A12_F53_I1 strains and their
corresponding proteomics shifts on glucose minimal medium with respect to IPL400.

| | | | | | |
|---|---|---|---|---|---|
| | | | | Sequence | Proteome |
| # | Position | Gene | Details | change | change |

A10_F63_I1

| | | | | | |
|---|---|---|---|---|---|
| 1 | 1,386,816 | oprD/PP_1207 | intergenic (−360/−331) | C→T | NS/NS |
| 2 | 1,812,462 | PP_1615/frmA | intergenic (−45/−60) | A→G | ND/Up |
| 3 | 1,888,423 | PP_1695 | L617R (CTG→CGG) | A→C | NS |
| 4 | 2,443,213 | topA | L784Q (CTG→CAG) | T→A | NS |
| 5 | 3,866379 | PP_3415 | G167R (GGC→CGC) | C→G | Down |
| 6 | 4,586,057 | PP_4061/PP_4063 | intergenic (+140/+75) | +C | ND/Up |
| 7 | 4,808,180 | PP_4234/dsbD-II | intergenic (−289/−265) | 2 bp→CG | NS |
| 8 | 6,049,015 | rpoZ | coding (200/264 nt) | +C | NS |

A12_F53_I1

| | | | | | |
|---|---|---|---|---|---|
| 1 | 1,386,816 | oprD/PP_1207 | intergenic (−360/−331) | C→T | NS/NS |
| 2 | 1,591,652 | PP_1395 | L81R (CTG→CGG) | A→C | NS |
| 3 | 1,812,462 | PP_1615/frmA | intergenic (−45/−60) | A→G | ND/Up |
| 4 | 1,832,579 | PP_1635 | A190T (GCG→ACG) | G→A | NS |
| 5 | 1,851,825 | relA | coding (911-922/2241 | Δ12 bp | NS |
| | | | nt) | | |
| 6 | 2,481,262 | PP_2175 | F29V (TTC→GTC) | A→C | ND |
| 7 | 3,410,136 | PP_3024 | coding (167/1197 nt) | Δ1 bp | Down |
| 8 | 3,866,001 | PP_3415 | P293S (CCA→TCA) | G→A | Down |
| 9 | 3,866,742 | PP_3415 | V46I (GTC→ATC) | C→T | |
| 10 | 4,586,057 | PP_4061/PP_4063 | intergenic (+140/+75) | +C | ND/Up |

NS: No Significant difference,
ND: Not Detected

### Example 2

### Evolved Pseudomonas putida KT2440 with Enhanced Tolerance to Isoprenol

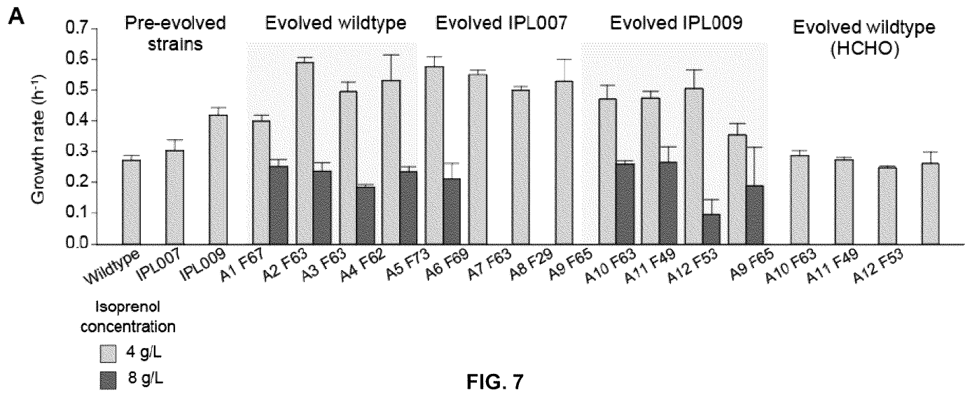

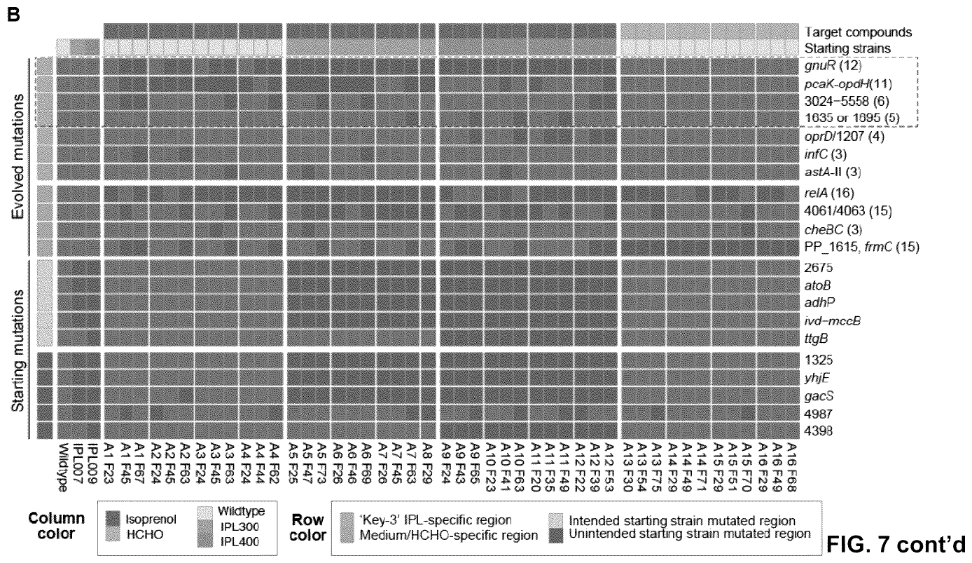

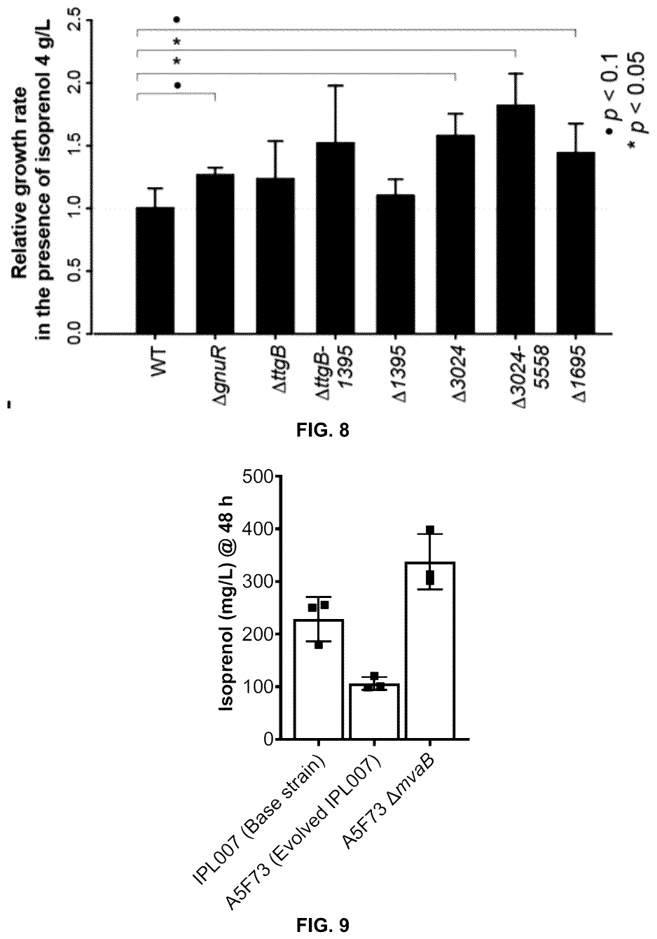

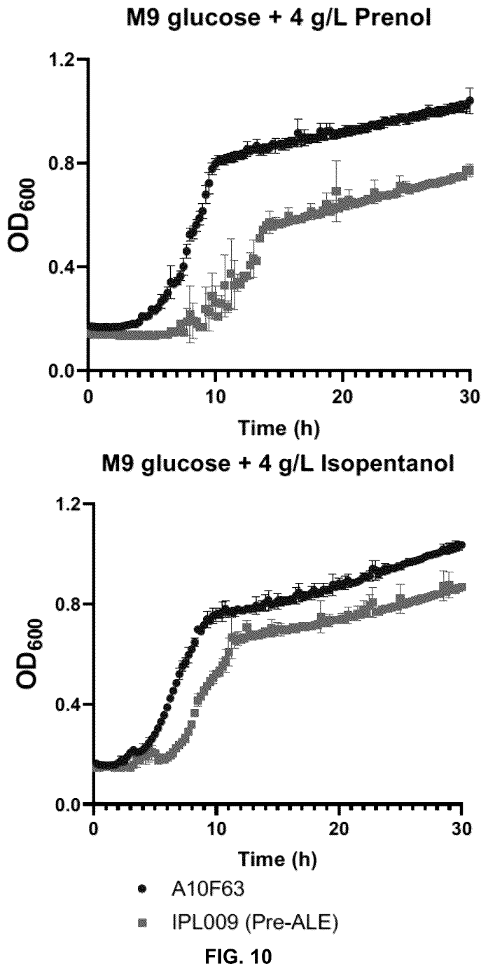

Isoprenol-tolerant isolates are generated using three starting strains (the wildtype P. putida KT2440, IPL300, IPL400), and demonstrated isoprenol production (FIG. 6). FIG. 7 shows the growth profiles of the wildtype KT2440 and evolved isolates. FIG. 8 shows the relative growth rates of the reverse-engineered strains to investigate the effect of mutated regions. Dot (⋅) and * indicates p<0.1, p<0.05, respectively. FIG. 9 shows the evolved strain A5F73 can be rationally engineered for enhancement in isoprenol titers. FIG. 10 shows the evolved strain A10F63 shows improved growth in the presence of 4 g/L prenol and isopentanol on glucose minimal medium.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified microbial strain capable of growing or dividing in a medium comprising equal to or more than about 4.5 g/L isoprenol, or having an increased tolerance to isoprenol compared to a unmodified microbial strain, wherein the microbial strain comprises one or more mutations from the following gene/genetic region: gnuR (PP_3415), pcaK (PP_1376)-opdH, PP_3024-PP_5558, erdR (PP_1635) or mxtR (PP_1695), oprD (PP_1206)/PP_1207, infC (PP_2466), astA-II (PP_4480), topA (PP_2139), PP_2402-calA (PP_2426), PP_3595-PP_3610, rpoZ (PP_5301), and/or fleQ, and any gene/genetic region described herein, such as in Table 1.

2. The genetically modified microbial strain of claim 1, wherein the microbial strain is a Pseudomonas, Azotobacter, Mesophilobacter, Oblitimonas, Permianibacter, Rugamonas, or Thiopseudomonas cell.

3. The genetically modified microbial strain of claim 2, wherein Pseudomonas cell is P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida.

4. The genetically modified microbial strain of claim 1, wherein the microbial strain is capable of growing or dividing in a medium comprising equal to or more than about 4.5 g/L, 5.0 g/L, 5.5 g/L, 6.0 g/L, 6.5 g/L, 7.0 g/L, 7.5 g/L, 8.0 g/L isoprenol, or a concentration within a range of any two preceding values.

5. The genetically modified microbial strain of claim 1, wherein the microbial strain is genetically modified to produce isoprenol or have an increased production of isoprenol

6. A method for a genetically modified bacterial host cell producing indigoidine, comprising (a) providing a genetically modified bacterial host cell of claim 1, (b) culturing or growing the host cell in a suitable culture or medium such that indigoidine is produced, and (c) optionally extracting or separating the indigoidine from the rest of the culture or medium, and/or host cell.

7. A method for producing isoprenol comprising: (a) providing a genetically modified microbial strain of claim 1, and (b) culturing or growing step the genetically modified microbial strain in a suitable medium to produce isoprenol.

8. The method of claim 7, wherein the genetically modified microbial strain produces isoprenol such that the concentration of isoprenol in the medium is equal to or more than about 4.5 g/L, 5.0 g/L, 5.5 g/L, 6.0 g/L, 6.5 g/L, 7.0 g/L, 7.5 g/L, 8.0 g/L isoprenol, or a concentration within a range of any two preceding values.

9. The method of claim 7, wherein the method further comprises separating or recovering isoprenol from the medium.


SEQUENCE LISTING (20 sequences)

SEQ ID NO: 1 - DNA, 20 bp, Synthetic construct
  source 1..20: mol_type = other DNA
cgaccaattgctcaaagcct

SEQ ID NO: 2 - DNA, 28 bp, Synthetic construct
  source 1..28: mol_type = other DNA
catgtatactaaactcacaaattagagc

SEQ ID NO: 3 - DNA, 50 bp, Synthetic construct
  source 1..50: mol_type = other DNA
gctctaatttgtgagtttagtatacatgtctgcaggaacaggtacatcac

SEQ ID NO: 4 - DNA, 44 bp, Synthetic construct
  source 1..44: mol_type = other DNA
agaggctttgagcaattggtcggaattccctacgtgccatgttg

SEQ ID NO: 5 - DNA, 50 bp, Synthetic construct
  source 1..50: mol_type = other DNA
gctctaatttgtgagtttagtatacatgatccataccccggatttcatcg

SEQ ID NO: 6 - DNA, 42 bp, Synthetic construct
  source 1..42: mol_type = other DNA
aggctttgagcaattggtcggaattccctacgtgccatgttg

SEQ ID NO: 7 - DNA, 50 bp, Synthetic construct
  source 1..50: mol_type = other DNA
gctctaatttgtgagtttagtatacatgattacaggctcctcggtattgc

SEQ ID NO: 8 - DNA, 42 bp, Synthetic construct
  source 1..42: mol_type = other DNA
aggctttgagcaattggtcgactcacacctgatgcgtatctc

SEQ ID NO: 9 - DNA, 43 bp, Synthetic construct
  source 1..43: mol_type = other DNA
aggctttgagcaattggtcgtatctacgtaagccatagccgcc

SEQ ID NO: 10 - DNA, 70 bp, Synthetic construct
  source 1..70: mol_type = other DNA
gatccgaatttctactgttgtagatggcgttcctgcccatctacccaaataaaacgaaaggctcagtcga

SEQ ID NO: 11 - DNA, 70 bp, Synthetic construct
  source 1..70: mol_type = other DNA
tcgactgagcctttcgttttatttgggtagatgggcaggaacgccatctacaacagtagaaattcggatc

SEQ ID NO: 12 - DNA, 91 bp, Synthetic construct
  source 1..91: mol_type = other DNA
aggtataatggatccgaatttctactgttgtagatagtgccgactcgccggaatctatttcaaataaaacgaaaggctcagtcgaaagact

SEQ ID NO: 13 - DNA, 91 bp, Synthetic construct; identical to SEQ ID NO: 12
  source 1..91: mol_type = other DNA

SEQ ID NO: 14 - DNA, 90 bp, Synthetic construct
  source 1..90: mol_type = other DNA
aggtataatggatccgaatttctactgttgtagatgcgaagccatccacaatgtcaatttcaaataaaacgaaaggctcagtcgaaagac

SEQ ID NO: 15 - DNA, 90 bp, Synthetic construct
  source 1..90: mol_type = other DNA
aggtataatggatccgaatttctactgttgtagatgtgtcccccaagtgggtgccaaaataaaacgaaaggctcagtcgaaagactgggc

SEQ ID NO: 16 - DNA, 87 bp, Synthetic construct
  source 1..87: mol_type = other DNA
tcgttgtccagcgggctgatcgccgtggtcgccctggcctataaccggtcaagcctgcggccttgcgcgccctgctcaatcgccatc

SEQ ID NO: 17 - DNA, 90 bp, Synthetic construct
  source 1..90: mol_type = other DNA
tgttaaggtggcgggatccaatcgccctgctgacgaggtgtgtgcaacctcatgtcccaagtctttgatgccagcgcattcctggcgacc

SEQ ID NO: 18 - DNA, 90 bp, Synthetic construct
  source 1..90: mol_type = other DNA
cacgcccctgcagggcgccgatatgactagggaagttgctattgctgtttgggctgggtgttcgccttgggggttgcgtttgggcggggt

SEQ ID NO: 19 - DNA, 90 bp, Synthetic construct
  source 1..90: mol_type = other DNA
gtatgcgtgccccgctccatccaataaaaacaaggtgaaaaggccgctgttccggccgcttcgcggatcaacccgcgaagcggccaaccg

SEQ ID NO: 20 - DNA, 90 bp, Synthetic construct
  source 1..90: mol_type = other DNA
ccctgaacaacaaagacaaccgctgaacgccacaggaaacccgccgtcacacgggtgtcacattggtgtgggtgaagtgttacctcccgc